(19) United States
(12) Patent Application Publication
(10) Pub. No.: US 2005/0287668 A1
(43) Pub. Date: Dec. 29, 2005

(54) RNA interference compositions and screening methods for the identification of novel genes and biological pathways

(75) Inventor: Robert E. Finney, Shoreline, WA (US)

Correspondence Address: DONALD W. WYATT;CELL THERAPEUTICS, INC., 501 ELLIOTT AVENUE WEST, #400, SEATTLE, WA, 98119, US

(73) Assignee: Cell Therapeutics, Inc. (CTI)

(21) Appl. No.: 10/976,467

(22) Filed: Oct. 29, 2004

Related U.S. Application Data
(60) Provisional application No. 60/517,411, filed on Nov. 4, 2003.

(51) Int. Cl. C12N 15/85

(52) U.S. Cl. 435/455

(57) ABSTRACT

The present invention provides compositions and methods for enhancing RNA interference and facilitating the use of long RNA interference molecules. Accordingly, the invention includes a variety of novel applications of RNA interference, including methods related to screening RNA interference molecules using reporter genes to identify biological pathways, genes, therapeutic compounds and biomarkers.

### BACKGROUND OF THE INVENTION

1. Field of the Invention

The present invention relates generally to the field of post-transcriptional gene silencing. More particularly, the present invention relates to methods and compositions for enhancing RNA interference-mediated silencing of gene expression. In addition, the invention relates to novel reporter systems for the screening of RNA interference reagents to identify biological pathways, genes, therapeutic compounds, and biomarkers.

2. Description of the Related Art

A variety of different and complementary approaches have been taken to identify novel therapeutic compounds. For example, cell-based assays have been performed to identify chemicals that produce a desired therapeutic effect on diseased cells, and chemical diversity libraries have been screened to identify chemical inhibitors and activators of gene expression. While these approaches may yield promising therapeutic candidates, their usefulness is limited, since they do not reveal the direct target or biological pathways targeted by the identified chemical entities. Accordingly, there is a need in the art for novel screening methods that identify these pathways and gene targets.

RNA interference (RNAi) is a biological process that involves sequence-specific mRNA degradation that is mediated by short interfering RNA (siRNA) molecules generated from the cleavage of dsRNA homologous to the gene targeted for silencing. The mechanism of RNAi-mediated specific gene silencing was first discovered in C. elegans and has also been found in other organisms, including Drosophila, hydra, zebrafish, and trypanasomes.

While the exact mechanism behind RNA interference is still not entirely understood, it appears that a dsRNA is processed into 20-25 nucleotide short interfering RNAs (siRNAs) by an Rnase III-like enzyme called Dicer. The siRNAs assemble into endoribonuclease-containing complexes known as RNA-induced silencing complexes (RISCs). The siRNA strands are then unwound to form activated RISCs, and the siRNA strands subsequently guide the RISCs to complementary RNA molecules, where they cleave and destroy the cognate RNA (discussed in Bass, B., NATURE 411:428-429 (2001) and Sharp, P. A., GENES DEV. 15:485-490 (2001)).

Although the phenomenon of RNAi was first characterized in C. elegans and Drosophila, RNAi has also been demonstrated to work in mammalian cells (Wianny, F. and Zernica-Goetz, M., (2000), NATURE CELL BIOLOGY Vol 2., 70-75. However, in most mammalian cells, introduction of long dsRNA molecules causes nonspecific suppression of gene expression, as opposed to gene-specific suppression seen in other organisms (Bass, B. L., (2001) NATURE 411:428-429). This suppression has been attributed to an antiviral response, which takes place through one of two pathways. In one pathway, long dsRNAs activate double-stranded RNA-activated protein kinase R (PKR). Activated PKR phosphorylates and inactivates the translation initiation factor, eIF2a, leading to repression of translation (Manche, L. et al., (1992) MOL. CELL. BIOL. 12:5238-5248). In the other pathway long dsRNAs activate Rnase L, which leads to nonspecific RNA degradation.

In an effort to circumnavigate this nonspecific suppression, researchers have taken the approach of introducing short siRNAs, as opposed to longer dsRNA molecules, into mammalian cells and have demonstrated that the introduction of certain siRNAs leads to the targeted degradation of corresponding mRNAs (see, e.g., Elbashir, S. M., et al., (2001) NATURE 411:494-498). Unfortunately, however, the identification of specific siRNAs that lead to suppression of any particular target gene has proven to be difficult and laborious, since not all siRNAs homologous to a target gene are effective in mediating suppression, and it is difficult to accurately and reliably predict which siRNAs will be the most effective. Accordingly, there is a need in the art for methods and compositions for enhancing RNAi in mammalian cells.

The present invention meets these needs by providing novel compositions and methods for enhancing RNAi, as well as related methods for screening RNAi reagents and chemical entities, which identify genes and biological pathways associated with normal and disease-related cellular processes, as well as the mechanism of action of therapeutic chemical entities.

### BRIEF SUMMARY OF THE INVENTION

The present invention provides compositions and methods related to RNAi, including compositions and methods designed to enhance RNAi, which may be used in a variety of application, including methods related to screening RNAi reagents to identify novel genetic pathways, genes and therapeutic compounds, and biomarkers.

In a first embodiment, the invention includes compositions nad methods for reducing non-specific gene suppression induced by RNAi reagents, including long RNAi reagents.

In one embodiment, the invention provides a method of reducing nonspecific suppression of gene expression in response to an introduced RNAi reagent or double-stranded polynucleotide that includes introducing an agent that attenuates a pathway of nonspecific suppression into a cell and introducing an RNAi molecule that induces nonspecific suppression of gene expression into the cell, wherein said agent reduces nonspecific suppression of gene expression induced by said double-stranded polynucleotide. The RNAi molecule may induce nonspecific suppression of gene expression in the same or a different cell. For example, the RNAi molecule may induce nonspecific gene suppression in the absence in a cell of an agent that attenuates a pathway of nonspecific suppression. In certain embodiments, the pathway of nonspecific suppression is the PKR pathway or the RNase L pathway. Accordingly, in certain embodiments, the agent alters the activity of a component of the PKR pathway or the RNase L pathway, or both. In particular embodiments, the agent reduces the activity of PKR or increases the activity of elongation initiation factor 2a.

In certain embodiments of the above method, the agent is a knockout reagent, such as targeting vectors and replacement vectors.

In other related embodiments, the agent is a knockdown reagent, such as antisense RNA, ribozymes, and RNAi molecules. In particular embodiments, RNAi molecules include RNA:RNA hybrids, sense DNA:antisense RNA hybrids, sense RNA:antisense DNA hybrids, and DNA:DNA hybrids.

In further related embodiments, the agent is a mutant or a dominant negative.

In particular embodiments of methods and compositions of the invention related to RNAi molecules, the RNAi molecule is at least 30 nucleotides in length, at least 50 nucleotides in length, at least 100 nucleotides in length, at least 200 nucleotides in length, at least 500 nucleotides in length, or at least 1000 nucleotides in length. In one embodiment, the RNAi reagent comprises a full length cDNA sequence.

In one embodiment, the invention includes a composition adapted for reducing nonspecific suppression of gene expression in response to an introduced double-stranded polynucleotide, wherein said composition comprises an agent that attenuates a pathway of nonspecific suppression, including the pathways and agents described above.

In various embodiments related to cells, the cell is a eukaryotic cell, a mammalian cell, a human cell, or a murine cell.

In one embodiment, the invention includes a mammalian cell adapted for reducing nonspecific suppression of gene expression in response to an introduced double-stranded polynucleotide, wherein the cell comprises an attenuated pathway of nonspecific suppression. In various embodiments, the cell comprises an agent that attenuates a pathway of nonspecific suppression, such as a knockout or knockdown reagent, including those described above. In other embodiments, the agent reduces the activity of PKR. In one embodiment, the agent reduces expression of PKR.

In particular embodiment, the cell comprises a polynucleotide that expresses the knockdown reagent. The polynucleotide may comprise an inducible promoter operably linked to a sequence that expresses the knockdown reagent. The polynucleotide may be a recombinant expression construct.

In other related embodiments, the cell comprises a disrupted gene, wherein the gene is a component of a pathway of nonspecific suppression, such as either the PKR or RNAse L pathway. In one embodiment, the gene encodes PKR.

In one embodiment, the cell further comprises an exogenous polynucleotide comprising a sequence that encodes a polypeptide encoded by the disrupted gene or a variant thereof. The exogenous polynucleotide may further comprise an inducible promoter operably linked to the sequence that encodes a polypeptide encoded by the disrupted gene or a variant thereof.

In certain embodiments of cells of the invention, a cell comprises a marker or reporter gene. In particular embodiments, the reporter gene is selected from the group consisting of: alkaline phosphatase, green fluorescent protein, chloramphenicol acetyltransferase, b-galactosidase, and other reporters. In one embodiment, expression of the reporter gene is regulated by an operably linked exogenous polynucleotide sequence. In a particular embodiment, the exogenous polynucleotide sequence comprises a regulatory element of a gene, which may be a mammalian gene, such as a human gene. In various related embodiment, the gene is an oncogene, tumor suppressor gene, cytokine gene, or apoptosis gene. The gene may be associated with human disease, such as a cancer.

In particular embodiments, cells of the invention comprise an RNAi molecule. In particular embodiments, the RNAi molecule is between 16 and 30 nucleotides in length, at least 30 nucleotides in length, at least 50 nucleotides in length, at least 100 nucleotides in length, at least 200 nucleotides in length, at least 500 nucleotides in length, or at least 1000 nucleotides in length. In one embodiment, the RNAi molecule comprises a full length cDNA sequence. In one embodiment, the sense strand of the RNAi molecule comprises at least 18 contiguous nucleotides of a PKR cDNA.

In related embodiments, the invention includes libraries and arrays of RNAi reagents and cells.

In one embodiment, the invention includes a library or array of cells of the invention, wherein the library comprises a plurality of RNAi molecules. In certain embodiment, the array comprises discrete identifiable locations, and wherein a plurality of the locations comprise one or more cells of the invention.

In one particular embodiment, the array comprises discrete identifiable locations, wherein a discrete location comprises one or more cells of the invention, wherein the cells at a discrete location comprise the same RNAi molecule, and wherein the cells at different discrete locations comprise a different RNAi molecule. In certain embodiment, each of the different RNAi molecules is capable of reducing expression of a different gene.

In another embodiment, the invention includes methods and compositions for performing RNAi using long RNAi reagents.

In one embodiment, the invention includes a method of reducing expression of a gene, comprising introducing into a cell an RNAi molecule that reduces expression of the gene. In particular embodiments, the RNAi molecule is at least 30 nucleotides in length, at least 50 nucleotides in length, at least 100 nucleotides in length, at least 200 nucleotides in length, at least 500 nucleotides in length, at least 1000 nucleotides in length, or a full length cDNA sequence. In various embodiments, RNAi molecules are RNA:RNA hybrids, sense DNA:antisense RNA hybrids, sense RNA:antisense DNA hybrids, or DNA:DNA hybrids. In one embodiment, the RNAi molecule reduces expression of an oncogene, tumor suppressor gene, cytokine gene, or apoptosis gene.

In a related embodiment, the invention includes a cell comprising an RNAi molecule that reduces expression of a gene. In certain embodiments, the RNAi molecule is at least 30 nucleotides in length, at least 50 nucleotides in length, at least 100 nucleotides in length, at least 200 nucleotides in length, at least 500 nucleotides in length, at least 1000 nucleotides in length, or a full length cDNA sequence. The RNAi molecule may be any form, including RNA:RNA hybrids, sense DNA:antisense RNA hybrids, sense RNA:antisense DNA hybrids, and DNA:DNA hybrids. In certain embodiment, the RNAi molecule reduces expression of an oncogene, tumor suppressor gene, cytokine gene, or apoptosis gene. In one embodiment, the cell further comprises a reporter gene, including, but not limited to, any of those described above. Expression of the reporter gene may be regulated by an operably linked exogenous polynucleotide sequence, which may comprise a regulatory element of a gene, such as a mammalian gene or a human gene. In particular embodiment, the gene is an oncogene, tumor suppressor gene, cytokine gene, or apoptosis gene. In one particular embodiment, the gene is associated with a human disease, such as a cancer, for example.

The invention further includes libraries and arrays of cells of the invention, wherein the library or array comprises a plurality of RNAi molecules.

In one embodiment, the array comprises discrete identifiable locations and a plurality of the locations comprise one or more cells of the invention. In certain embodiments, the array comprises discrete identifiable locations, wherein a discrete location comprises one or more cells, wherein the cells at a discrete location comprise the same RNAi molecule, and wherein the cells at different discrete locations comprise a different RNAi molecule. In a related embodiment, each of the different RNAI molecules reduces expression of a different gene.

In yet another embodiment, the invention provides a method of determining a biological function of a gene, which includes introducing an RNAi molecule that reduces expression of a gene into a cell, wherein the RNAi molecule is at least 30 nucleotides in length, and comparing a biological trait of the cell of step (a) to that of a control. In one embodiment, the control is a cell wherein an RNAi molecule is not introduced. In particular embodiments, the cell is a cell of the invention.

In a related embodiment, the invention includes a method of determining a biological function of a gene, which includes providing a cell, introducing an RNAi molecule that reduces expression of a gene to the cell, wherein the RNAi molecule is at least 30 nucleotides in length, and comparing a biological trait of the cell before and after introduction of the RNAi molecule into the cell. In particular embodiments, the cell is a cell of the invention.

The invention further includes a method of determining the effect of reducing the expression of a gene on the expression of a reporter gene, which includes providing a cell comprising a reporter gene, introducing an RNAi molecule that reduces expression of a gene to the cell, wherein the RNAi molecule is at least 30 nucleotides in length, and comparing expression levels of the reporter gene before and after introduction of the RNAi molecule to the cell.

In certain embodiments, the cell further comprises an agent that attenuates a pathway of nonspecific suppression. In one embodiment, the agent reduces expression of PKR.

In other embodiments, expression of the reporter gene is regulated by an operably linked exogenous polynucleotide sequence, which may comprise a regulatory element of a gene. In particular embodiments, the gene is a mammalian gene or a human gene. Further, in some embodiments, the gene is an oncogene, tumor suppressor gene, cytokine gene, or apoptosis gene. The gene may be associated with a human disease, which, in one embodiment, is a cancer.

In another embodiment, the invention includes a method of identifying a gene associated with a biological attribute, which includes providing an array of cells of the invention, identifying a cell having an altered biological attribute as compared to a control cell, determining a sequence of the RNAi molecule in the cell identified, and identifying a gene having the sequence determined. In one embodiment, the cells further comprise an agent that attenuates a pathway of nonspecific suppression, including any pathway of agent described above.

In yet another related embodiment, the invention includes a method of identifying a gene that alters the expression of a reporter gene, which includes providing an array of cells of the invention, identifying a cell of the array having altered expression of a reporter gene as compared to a control cell, determining a sequence of the RNAi molecule in the cell identified in step (b); and identifying a gene having the sequence determined. In one embodiment, expression of the reporter gene is regulated by an operably linked exogenous polynucleotide sequence, which may comprise a regulatory element of a gene In certain embodiments, the gene is a mammalian or human gene. In certain embodiments, the gene is an oncogene, tumor suppressor gene, cytokine gene, differentiation gene or apoptosis gene. The gene may be associated with a human disease, which may be a cancer. In related embodiment, the gene has been implicated in human disease through the use of expression arrays, proteomics, or bioinformatics.

In another embodiment, the invention includes a method of identifying a gene associated with growth or viability of tumor cells, which includes providing an array of cells of the invention, wherein the cells are tumor cells, identifying a cell having altered growth or viability as compared to a control cell, determining a sequence of the RNAi molecule in the cell identified, and identifying a gene having the sequence determined. The cells may be any cell of the invention, including those described above. In one embodiment, the cells further comprise an agent that attenuates a pathway of nonspecific suppression.

In a related embodiment, the invention includes a method of identifying a gene associated with tumor cell sensitivity to a chemical agent, which includes providing an array of cells of the invention, wherein the cells are tumor cells, treating the cells with a chemical agent, identifying a cell having altered sensitivity to the chemical agent as compared to a control cell, determining a sequence of the RNAi molecule in the cell identified, and identifying a gene having the sequence determined. In related embodiments, the cells further comprise an agent that attenuates a pathway of nonspecific suppression. In other related embodiments, the chemical agent is a drug or drug candidate. In particular embodiment, the sensitivity is proliferation, apoptosis, senescence, or differentiation.

In another embodiment, the invention includes a method of identifying a gene that alters expression of a gene, which includes providing an array of cells of the invention, identifying a cell having altered expression of a first gene as compared to a control cell, determining a sequence of the RNAi molecule in the cell identified, and identifying a second gene having the sequence determined, wherein the second gene alters expression of the first gene. In one embodiment, the first gene is an oncogene, tumor suppressor gene, cytokine gene, differentiation gene or apoptosis gene. In one embodiment, the cells further comprise an agent that attenuates a pathway of nonspecific suppression.

In a further embodiment, the invention includes methods and compositions for screening libraries of RNAi reagents to identify an RNAi reagent having a desired effect on a cell or gene.

### DETAILED DESCRIPTION OF THE INVENTION

The present invention provides methods and compositions useful in enhancing RNAi in mammalian cells. In one aspect, the invention provides methods and compositions for reducing dsRNA-induced nonspecific suppression of gene expression, thus facilitating the use of long RNAi molecules in performing RNAi of specific target genes in mammalian cells. Accordingly, the invention includes both novel methods and compositions for reducing nonspecific suppression and novel methods and compositions for performing RNAi to reduce expression of target genes. In a second related aspect, the invention provides methods and compositions for performing RNAi using long RNAi molecules, including libraries and arrays of cells comprising a plurality of long RNAi molecules, in the presence or absence of an agent capable of reducing nonspecific suppression of gene expression. In addition, the invention includes a variety of novel applications of this technology, including, e.g., in identifying gene function and in screening for modulators of gene function and disease, e.g., using reporter cells.

A. Methods and Compositions for Reducing Nonspecific Gene Suppression

In one aspect, the present invention provides methods and compositions for reducing or attenuating nonspecific gene suppression. These methods typically involve altering the activity of a molecule that mediates or effects nonspecific gene suppression. As described above, two pathways of nonspecific suppression have been characterized. In one pathway, long dsRNAs activate a protein kinase, PKR. Activated PKR phosphorylates and inactivates the translation initiation factor, eIF2a, leading to repression of translation (Manche, L. et al., (1992) MOL. CELL. BIOL. 12:5238-5248). In the other pathway, long dsRNAs activate Rnase L, which leads to nonspecific RNA degradation. Accordingly, the present invention contemplates altering or attenuating the activity of one or more molecules involved in either or both pathways.

While altering the activity of a molecule involved in nonspecific gene suppression typically involves reducing the activity of the molecule, e.g., where the molecule is a mediator of effecter of nonspecific gene suppression, the invention also contemplates other methods of altering the activity of a molecule involved in nonspecific gene suppression, including, e.g., increasing the activity of a molecule displaying reduced activity in response to a pathway of nonspecific gene suppression.

It is understood that the activity of a molecule may be altered by any of a variety of means, including, but not limited to, increasing or reducing expression of mRNA encoding the molecule, altering the sequence of a molecule to increase or decrease the molecule's biological, enzymatic, or functional activity, or indirectly altering the activity of a molecule by altering the expression or activity of a molecule that acts in concert with said molecule, such as, e.g., a binding partner or a molecule that acts upstream of said molecule to regulate its activity or downstream of said molecule as a target or effecter of said molecule.

As noted above, the present invention may involve altering the activity of one or more molecules involved in or required for nonspecific gene suppression. Thus, in certain embodiments, the invention includes altering the activity of only one molecule involved in nonspecific gene suppression, such as, e.g., reducing expression of a critical mediator of nonspecific gene suppression. In other related embodiments, the invention includes altering the activity of two or more molecules associated with nonspecific gene suppression. In certain embodiments, these molecules may be involved in the same pathway of nonspecific gene suppression, while in other embodiments, it may be advantageous to alter the activity of molecules in two or more pathways of nonspecific gene suppression. Accordingly, in one embodiment, the present invention includes altering the activity of one or more molecules associated with one pathway of nonspecific gene suppression and also altering the activity of one or more molecules associated with a second pathways of nonspecific gene suppression. It is recognized that certain molecules may be associated with two or more pathways of nonspecific gene suppression, so it is possible to effect two or more pathways of nonspecific gene suppression by altering a single molecule associated with all the effected pathways.

1. Targets

The invention includes altering the activity of any molecule that plays a role in nonspecific gene suppression. For example, in certain embodiments, the molecule is an initiator of nonspecific gene suppression, and in other embodiments, the molecule is a mediator or effecter of nonspecific gene expression. In general, the activity of any molecule involved in nonspecific gene suppression may be altered according to the invention, so long as altering the activity of the molecule results in a decrease in nonspecific gene suppression induced by a long RNAi molecule. In certain embodiments, the decrease in nonspecific gene suppression is an at least 25% reduction, an at least 50% reduction, an at least 75% reduction, an at least 90% reduction, or an at least 99% reduction. In one embodiment, the decrease in nonspecific gene suppression is a 100% reduction. Nonspecific gene suppression may be measured as the reduction in transcription of a non-targeted gene, or the average reduction of two of more non-targeted genes, in response to the introduction of a long RNAi molecule, such as a long double-stranded RNA, into a cell.

Two major pathways of nonspecific gene suppression have been identified, including the PKR pathway and the RNase L pathway. Accordingly, in certain embodiments, the invention involves altering the activity of a component of one or both of these pathways. Further details regarding these pathways and the various molecules involved in these pathways are provided below.

a. PKR Pathway

The PKR protein kinase is among the best-studied effectors of the host interferon (IFN)-induced antiviral and antiproliferative response system (reviewed in Tan, S.-L., and Katze, M. G. (1999) J. Interferon Cytokine Res. 19, 545-556). In response to stress signals, including virus infection, the normally latent PKR becomes activated through autophosphorylation and dimerization and phosphorylates the eIF2alpha translation initiation factor subunit, leading to an inhibition of mRNA translation initiation. PKR is ubiquitously expressed but is normally inactive, presumably because the ATP-binding site or the catalytic domain of PKR is masked by intramolecular interactions (Wu, S., and Kaufman, R. J. (1996) J. Biol. Chem. 271: 756-1763, Carpick, B. W., Graziano, V., Schneider, D., Maitra, R. K., Lee, X., and Williams, B. R. G. (1997) J. Biol. Chem. 272, 9510-9516). Upon binding to dsRNA, or to RNA with secondary structures similar to viral replicative intermediates, PKR is autophosphorylated on multiple serine and threonine residues, which may induce a conformational change that leads to the disclosure of the ATP-binding site and/or the catalytic domain. This is followed by PKR dimerization, which is thought to promote the intermolecular autophosphorylation of PKR molecules, resulting in maximal activation of the enzyme (Kostura, M., and Mathews, M. B. (1989) Mol. Cell. Biol. 9, 1576-1586). Binding to dsRNA may also serve to recruit PKR molecules to the ribosomes for localized action, where phosphorylation of eIF2x by PKR leads to a block in global protein synthesis.

In addition to inhibiting translation initiation through the phosphorylation of the alpha subunit of the initiation factor eIF-2 (eIF-2 alpha), PKR also controls the activation of several transcription factors such as NF-kappa B, p53, or STATs. PKR also mediates apoptosis induced by many different stimuli, such as treatment with LPS, TNF-alpha, viral infection, or serum starvation. The mechanism of apoptosis induction by PKR involves phosphorylation of eIF-2 alpha and activation of NF-kappa B. In this way, expression of different genes is regulated by PKR. Among the genes upregulated in response to PKR are Fas, Bax and p53. The pathway of PKR-induced apoptosis involves FADD activation of caspase 8 by a mechanism independent of Fas and TNFR.

While numerous virally encoded or modulated proteins that bind and inhibit PKR during virus infection have been studied, little is known about the cellular proteins that counteract PKR activity in uninfected cells. Overexpression of PKR in yeast also leads to an inhibition of eIF2alpha-dependent protein synthesis, resulting in severe growth suppression. Screening of a human cDNA library for clones capable of counteracting the PKR-mediated growth defect in yeast led to the identification of the catalytic subunit (PP1 (C)) of protein phosphatase 1 alpha. PP1 (C) reduced double-stranded RNA-mediated auto-activation of PKR and inhibited PKR transphosphorylation activities (Tan, S. L. et al., 2002 J BIOL CHEM. 277:36109-17). A specific and direct interaction between PP1 (C) and PKR was detected, with PP1 (C) binding to the N-terminal regulatory region regardless of the double-stranded RNA-binding activity of PKR. A consensus motif shared by many PP1(C)-interacting proteins was necessary for PKR binding to PP1(C). The PKR-interactive site was mapped to a C-terminal non-catalytic region that is conserved in the PP1(C)₂ isoform. Indeed, co-expression of PP1 (C) or PP1(C)₂ inhibited PKR dimer formation in Escherichia coli. Interestingly, co-expression of a PP1(C) mutant lacking the catalytic domain, despite retaining its ability to bind PKR, did not prevent PKR dimerization. These findings suggest that PP1(C) modulates PKR activity via protein dephosphorylation and subsequent disruption of PKR dimers.

According to the present invention, any of the above described components of the PKR pathway of nonspecific gene suppression may be altered to reduce nonspecific gene suppression in response to long RNAi molecules. In one particular embodiment, the activity of PKR is reduced. In another embodiment, the activity of eIF2a is increased. In other embodiments, the activity of NF-kappa B, p53, a STAT, Fas, Bax, p53 or caspase-8 is reduced. In another embodiment, the activity of a protein phosphatase 1alpha is increased. In certain embodiments, the activity of two or more molecules associated with nonspecific gene regulation are altered.

b. RNase L Pathway

The 2-5A system is an RNA degradation pathway that can be induced by the interferons (IFNs) (reviewed in Player, M. R. and Torrence, P. F., PHARMACOL THER. 1998 May; 78(2):55-113). Interferon treatment of cells leads to an increase in basal, but latent, levels of 2-5A-dependent RNase (RNase L) and the family of 2′-5′ oligoadenylate synthetases (OAS). Double-stranded RNA activates OAS. Activated OAS converts ATP into unusual short 2′-5′ linked oligoadenylates called 2-5A [ppp5′(A2′p5′)2A]. The 2-5A binds to and activates RNase L which cleaves single stranded RNA with moderate specificity for sites 3′ of UpUp and UpAp sequences, and thus leads to degradation of cellular rRNA. During apoptosis, generalized cellular RNA degradation, distinct from the differential expression of mRNA species that may regulate specific gene expression during apoptosis, has been observed. The mechanism of RNA breakdown during apoptosis has been commonly considered a non-specific event that reflects the generalized shut down of translation and homeostatic regulation during cell death.

Inhibition of RNase L, specifically with a dominant negative mutant, suppressed poly(I)Ypoly(C)-induced apoptosis in interferon-primed fibroblasts (Castellu, J. et al., BIOMED PHARMACOTHER. 1998; 52(9):386-90). Poliovirus, a picornovirus with a single-stranded RNA genome, causes apoptosis of HeLa cells. Expression of the dominant negative inhibitor of RNase L in HeLa prevented virus-induced apoptosis and maintained cell viability. Thus, reduction or inhibition of RNase L activity prevents apoptosis. Accordingly, the present invention includes agents capable of altering the activity of one or more components of the RNase L pathway, including, e.g., reducing the activity of RNase L or OAS.

2. Methods and Compositions

Altering the activity of a molecule involved in nonspecific gene suppression may be accomplished by any of a variety of means. For example, the activity of a molecule may be altered by increasing or decreasing expression of the gene encoding the molecule. Also, the activity of a molecule may be altered by disrupting its ability to interact with other components of its pathway. Examples of methods of reducing the expression of a molecule include, but are not limited to, knocking out all or a region of one or more alleles of a gene encoding the molecule and knocking down expression of a gene encoding a molecule. Methods of increasing the expression of a molecule, include, amongst others, introducing a transgene encoding the molecule into a cell. The skilled artisan would appreciate that there are a wide variety of methods of reducing the activity of molecule, including, e.g., reducing the expression or activity of a binding partner or functionally cooperating molecule and expressing a dominant negative inhibitor or the molecule. In addition, there are a wide variety of methods available for increasing the activity of a molecule, including, e.g., reducing the expression of an inhibitor or the molecule, increasing expression of a functionally cooperative binding partner of the molecule, or expressing a mutant form of the molecule that has increased activity. The activity of a molecule may be altered, i.e., increased or reduced, by at least 10%, at least 20%, at least 30%, at least 40%, at least 50%, at least 60%, at least 70%, at least 80%, at least 90%, or 100%. Increased or decreased in activity may be determined by a variety of means known and available in the art, depending upon the particular molecule affected. For example, where the molecule is a kinase, such as PKR, activity may be measured as enzymatic activity, including, e.g., the ability of a cellular extract to phosphorylate a substrate. Wherein activity is altered by altering expression of a gene encoding a molecule with altered activity, expression may be altered i.e., increased or reduced, by at least 10%, at least 20%, at least 30%, at least 40%, at least 50%, at least 60%, at least 70%, at least 80%, at least 90%, or 100%. Increased or decreased expression may be determined by a variety of means known and available in the art, including, e.g., polymerase chain reaction (PCR) employing primers specific for the gene and northern blot analysis using a probe specific for the gene.

In one particular embodiment of the invention, the activity of PKR is reduced by decreasing expression of PKR. This may be accomplished by any method available in the art. In certain embodiments, the activity of PKR is reduced by knocking out one or more alleles of a PKR gene. In other embodiments, the activity of PKR is reduced by knocking down expression of PKR using a knockdown reagent, including those described below. It is understood that constitutively decreasing expression of PKR may have deleterious effects on a cell. Accordingly, in certain embodiments, PKR expression is reduced transiently, for example, using an inducible knockdown reagent, or by knocking out or knocking down PKR expression in the presence of an inducible or tissue- or stage-specific promoter driving expression of a PKR gene. Methods of inducible knockout and knockdown are described in further detail infra. In another embodiment, the activity of PKR is reduced by mutating one or more alleles of PKR such that the autophosphorylation site in PKR is substituted by a different amino acid, which is not subject to autophosphorylation.

In another related embodiment of the invention, the activity of eIF2 alpha is increased by overexpressing eIF2 alpha. This may be accomplished by any of a variety of means, including, e.g., expressing eIF2 alpha from a transgene introduced into a cell. The transgene may be stably integrated into the cellular genome or transiently present in the genome. In a related embodiment, the activity of eIF2 alpha is increased by mutating one or more alleles of eIF2 alpha in the cell. For example, the PKR phosphorylation site on eIF2 alpha may be mutated, e.g., by substituting the amino acid residue that is phosphorylated by PKR with a different amino acid, which is not phosphorylated by PKR. In certain embodiments, the serine 52 residue of eIF2 alpha is substituted by a different residue, such as alanine.

Accordingly, the invention includes knockdown and knockdown reagents comprising at least a portion of a gene that encodes a molecule associated with nonspecific gene suppression. The portion of a gene may be a coding region, a non-coding region, or a region that includes both coding and noncoding regions. The skilled artisan can readily determine an appropriate region, depending in large part, upon the nature or type of specific knockout or knockdown reagent used. In addition, the length and percent identity of the region included in the knockout or knockdown reagent may be readily determined by the skilled artisan depending upon the nature or type of reagent used.

The methods and compositions described below are provided for exemplary purposes, and it is understood that the invention is not limited to these particular methods.

a. Knockout

In certain embodiments of the present invention, expression of a molecule associated with nonspecific gene suppression is reduced by knocking out one or more alleles of a gene encoding the molecule. Accordingly, the invention includes knockout vectors directed to a gene encoding a molecule associated with nonspecific gene suppression, cells comprising a knocked out allele of such a gene, and animals comprising such a cell. It is understood that knockout vectors according to the invention include any vector capable of disrupting expression or activity of a gene encoding a molecule associated with nonspecific gene suppression, including, in certain embodiments, both gene trap and targeting vectors. In preferred methods, targeting vectors are used to selectively disrupt a gene encoding a molecule associated with nonspecific gene suppression. Knockout vectors of the invention include those that alter gene expression, for example, by disrupting a regulatory element of a gene, including, e.g., inserting a regulatory element that reduces gene expression or deleting or otherwise reducing the activity of an endogenous element that positively affects transcription of the target gene. In other embodiments, knockout vectors of the invention disrupt, e.g., delete or mutate, the 5′ region, 3′ region or coding region of a gene. In some embodiments, knockout vectors delete a region or the entirety of the coding region of a gene. In certain embodiments, knockout vectors delete a region of a gene, while in other embodiments, they insert exogenous sequences into a gene. Of course, in certain embodiments, including those using replacement vectors, knockout vectors both remove a region of a gene and introduce an exogenous sequence.

Targeting vectors of the invention include all vectors capable of undergoing homologous recombination with an endogenous gene, including replacement vectors. Targeting vectors include all those used in methods of positive selection, negative selection, positive-negative selection, and positive switch selection. Targeting vectors employing positive, negative, and positive-negative selection are well known in the art and representative examples are described in Joyner, A. L., GENE TARGETING: A PRACTICAL APPROACH, 2nd ed. (2000) and references cited therein. Vectors employing positive switch selection methods are described in U.S. patent application Ser. No. 10/028,970, filed Dec. 28, 2001, which is hereby incorporated in its entirety. Essentially, positive switch selection methods involve replacing an original selection marker sequence of a gene trap construct with a reporter sequence and/or a new selection marker sequence.

b. Knockdown

According to the invention, the activity of a molecule associated with nonspecific gene suppression may be altered using a knockdown reagent that targets the molecule. Any knockdown reagent may be used according to the invention, including not limited to, (i) antisense sequences, (ii) catalytic RNAs (ribozymes), and (iii) RNAi molecules, including, for example, short interfering RNA (siRNA) and short hairpin RNA (shRNA), etc. Such knockdown reagents generally target a specific nucleotide sequence in genomic DNA or mRNA transcripts. Accordingly, in one embodiment of the invention, knockdown reagents comprise a polynucleotide sequence corresponding to a region of a target gene encoding a molecule associated with nonspecific gene suppression.

i. Antisense

Antisense oligonucleotides have been demonstrated to be effective and targeted inhibitors of protein synthesis, and, consequently, can be used to specifically inhibit protein synthesis by a targeted gene. The efficacy of antisense oligonucleotides for inhibiting protein synthesis is well established. For example, the synthesis of polygalactauronase and the muscarine type 2 acetylcholine receptor are inhibited by antisense oligonucleotides directed to their respective mRNA sequences (U.S. Pat. No. 5,739,119 and U.S. Pat. No. 5,759,829). Further, examples of antisense inhibition have been demonstrated with the nuclear protein cyclin, the multiple drug resistance gene (MDG1), ICAM-1, E-selectin, STK-1, striatal GABAA receptor and human EGF (Jaskulski et al., Science. 1988 Jun. 10; 240(4858):1544-6; Vasanthakumar and Ahmed, Cancer Commun. 1989;1 (4):225-32; Peris et al., Brain Res Mol Brain Res. 1998 Jun. 15; 57(2):310-20; U.S. Pat. No. 5,801,154; U.S. Pat. No. 5,789,573; U.S. Pat. No. 5,718,709 and U.S. Pat. No. 5,610,288). Furthermore, antisense constructs have also been described that inhibit and can be used to treat a variety of abnormal cellular proliferations, e.g. cancer (U.S. Pat. No. 5,747,470; U.S. Pat. No. 5,591,317 and U.S. Pat. No. 5,783,683).

Therefore, in certain embodiments, the present invention provides oligonucleotide sequences that comprise all, or a portion of, any sequence that is capable of specifically binding to a selected target polynucleotide sequence, or a complement thereof. In one embodiment, the antisense oligonucleotides comprise DNA or derivatives thereof. In another embodiment, the oligonucleotides comprise RNA or derivatives thereof. The antisense oligonucleotides may be modified DNAs comprising a phosphorothioated modified backbone. Also, the oligonucleotide sequences may comprise peptide nucleic acids or derivatives thereof. In each case, preferred compositions comprise a sequence region that is complementary, and more preferably, completely complementary to one or more portions of a target gene or polynucleotide sequence. Selection of antisense compositions specific for a given sequence is based upon analysis of the chosen target sequence and determination of secondary structure, Tₘ, binding energy, and relative stability. Antisense compositions may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences which are substantially complementary to 5′ regions of the mRNA. These secondary structure analyses and target site selection considerations can be performed, for example, using v.4 of the OLIGO primer analysis software and/or the BLASTN 2.0.5 algorithm software (Altschul et al., Nucleic Acids Res. 1997, 25(17):3389-402).

The use of an antisense delivery method employing a short peptide vector, termed MPG (27 residues), is also contemplated. The MPG peptide contains a hydrophobic domain derived from the fusion sequence of HIV gp41 and a hydrophilic domain from the nuclear localization sequence of SV40 T-antigen (Morris et al., Nucleic Acids Res. 1997 Jul. 15;25(14):2730-6). It has been demonstrated that several molecules of the MPG peptide coat the antisense oligonucleotides and can be delivered into cultured mammalian cells in less than 1 hour with relatively high efficiency (90%). Further, the interaction with MPG strongly increases both the stability of the oligonucleotide to nuclease and the ability to cross the plasma membrane.

ii. Ribozymes

According to another embodiment of the invention, ribozyme molecules are used to inhibit expression of a target gene or polynucleotide sequence. Ribozymes are RNA-protein complexes that cleave nucleic acids in a site-specific fashion. Ribozymes have specific catalytic domains that possess endonuclease activity (Kim and Cech, Proc Natl Acad Sci USA. 1987 December; 84(24):8788-92; Forster and Symons, Cell. 1987 Apr. 24;49(2):211-20). For example, a large number of ribozymes accelerate phosphoester transfer reactions with a high degree of specificity, often cleaving only one of several phosphoesters in an oligonucleotide substrate (Cech et al., Cell. 1981 December; 27(3 Pt 2):487-96; Michel and Westhof, J Mol. Biol. 1990 Dec. 5; 216(3):585-610; Reinhold-Hurek and Shub, Nature. 1992 May 14; 357(6374):173-6). This specificity has been attributed to the requirement that the substrate bind via specific base-pairing interactions to the internal guide sequence (“IGS”) of the ribozyme prior to chemical reaction.

At least six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme may be advantageous over many technologies, such as antisense technology (where a nucleic acid molecule simply binds to a nucleic acid target to block its translation), since the concentration of ribozyme necessary to affect inhibition of expression is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., Proc Natl Acad Sci U S A. 1992 Aug. 15; 89(16):7305-9). Thus, the specificity of action of a ribozyme is greater than that of an antisense oligonucleotide binding the same RNA site.

The enzymatic nucleic acid molecule may be formed in a hammerhead, hairpin, a hepatitis 6 virus, group I intron or RNaseP RNA (in association with an RNA guide sequence) or Neurospora VS RNA motif, for example. Specific examples of hammerhead motifs are described by Rossi et al. Nucleic Acids Res. 1992 Sep. 11;20(17):4559-65. Examples of hairpin motifs are described by Hampel et al., (Eur. Pat. Appl. Publ. No. EP 0360257), Hampel and Tritz, Biochemistry 1989 Jun. 13; 28(12):4929-33; Hampel et al., Nucleic Acids Res. 1990 Jan. 25; 18(2):299-304 and U.S. Pat. No. 5,631,359. An example of the hepatitis 6 virus motif is described by Perrotta and Been, Biochemistry. 1992 Dec. 1;31(47):11843-52; an example of the RNaseP motif is described by Guerrier-Takada et al., Cell. 1983 December; 35(3 Pt 2):849-57; Neurospora VS RNA ribozyme motif is described by Collins (Saville and Collins, Cell. 1990 May 18; 61(4):685-96; Saville and Collins, Proc Natl Acad Sci USA. 1991 Oct. 1; 88(19):8826-30; Collins and Olive, Biochemistry. 1993 Mar. 23; 32(11):2795-9); and an example of the Group I intron is described in (U.S. Pat. No. 4,987,071). Important characteristics of enzymatic nucleic acid molecules used according to the invention are that they have a specific substrate binding site which is complementary to one or more of the target gene DNA or RNA regions, and that they have nucleotide sequences within or surrounding that substrate binding site which impart an RNA cleaving activity to the molecule. Thus the ribozyme constructs need not be limited to specific motifs mentioned herein.

Ribozymes may be designed as described in Int. Pat. Appl. Publ. No. WO 93/23569 and Int. Pat. Appl. Publ. No. WO 94/02595, each specifically incorporated herein by reference and synthesized to be tested in vitro and in vivo, as described. Such ribozymes can also be optimized for delivery. While specific examples are provided, those in the art will recognize that equivalent RNA targets in other species can be utilized when necessary.

Ribozyme activity can be optimized by altering the length of the ribozyme binding arms or chemically synthesizing ribozymes with modifications that prevent their degradation by serum ribonucleases (see e.g., Int. Pat. Appl. PubI. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

iii. RNAi Molecules

RNA interference methods using RNAi molecules also may be used to disrupt the expression of a gene or polynucleotide of interest. While the first described RNAi molecules were RNA:RNA hybrids comprising both an RNA sense and an RNA antisense strand, it has now been demonstrated that DNA sense:RNA antisense hybrids, RNA sense:DNA antisense hybrids, and DNA:DNA hybrids are capable of mediating RNAi (Lamberton, J. S. and Christian, A. T., (2003) Molecular Biotechnology 24:111-119). Accordingly, the invention includes the use of RNAi reagents comprising any of these different types of double-stranded molecules. The description of RNAi reagents that follows describes double-stranded RNA (dsRNA) molecules, i.e., RNA:RNA hybrids, for exemplary purposes, but RNA sense:DNA antisense hybrids, DNA sense:RNA antisense and DNA hybrids are similarly included within the invention. In addition, it is understood that RNAi reagents may be used and introduced to cells in a variety of forms. Accordingly, as used herein, RNAi reagents encompasses any and all reagents capable of inducing an RNAi response in cells, including, but not limited to, double-stranded polynucleotides comprising two separate strands, i.e. a sense strand and an antisense strand, polynucleotides comprising a hairpin loop of complementary sequences, which forms a double-stranded region, e.g., shRNAi molecules, and expression vectors that express one or more polynucleotides capable of forming a double-stranded polynucleotide alone or in combination with another polynucleotide.

A dsRNA molecule that targets and induces degradation of an mRNA that is derived from a gene or polynucleotide of interest can be introduced into a cell. The exact mechanism of how the dsRNA targets the mRNA is not essential to the operation of the invention, other than the dsRNA shares sequence homology with the mRNA transcript. The mechanism could be a direct interaction with the target gene, an interaction with the resulting mRNA transcript, an interaction with the resulting protein product, or another mechanism. Again, while the exact mechanism is not essential to the invention, it is believed the association of the dsRNA to the target gene is defined by the homology between the dsRNA and the actual and/or predicted mRNA transcript. It is believed that this association will affect the ability of the dsRNA to disrupt the target gene. DsRNA methods and reagents are described in PCT applications WO 99/32619, WO 01/68836, WO 01/29058, WO 02/44321, WO 01/92513, WO 01/96584, and WO 01/75164, which are hereby incorporated by reference in their entirety.

In one embodiment of the invention, RNA interference (RNAi) may be used to specifically inhibit target nucleic acid expression. Double-stranded RNA-mediated suppression of gene and nucleic acid expression may be accomplished according to the invention by introducing dsRNA, siRNA or shRNA into cells or organisms. dsRNAs less than 30 nucleotides in length do not appear to induce nonspecific gene suppression, as described supra for long dsRNA molecules. Indeed, the direct introduction of siRNAs to a cell can trigger RNAi in mammalian cells (Elshabir, S. M., et al. Nature 411: 494-498 (2001)). Furthermore, suppression in mammalian cells occurred at the RNA level and was specific for the targeted genes, with a strong correlation between RNA and protein suppression (Caplen, N. et al., PROC. NATL. ACAD. SCI. USA 98:9746-9747 (2001)). In addition, it was shown that a wide variety of cell lines, including HeLa S3, COS7, 293, NIH/3T3, A549, HT-29, CHO-KI and MCF-7 cells, are susceptible to some level of siRNA silencing (Brown, D. et al. TECHNOTES 9(1):1-7, available at http://www.ambion.com/techlib/tn/91/912.html (Sep. 1, 2002)).

Structural characteristics of effective siRNA molecules have been identified. Elshabir, S. M. et al. (2001) NATURE 411:494-498 and Elshabir, S. M. et al., (2001), EMBO 20:6877-6888. Accordingly, one of skill in the art would understand that a wide variety of different siRNA molecules may be used to target a specific gene or transcript. In certain embodiments, siRNA molecules according to the invention are 16-30 or 18-25 nucleotides in length, including each integer in between. In one embodiment, an siRNA is 21 nucleotides in length. In certain embodiments, siRNAs have 0-7 nucleotide 3′ overhangs or 0-4 nucleotide 5′ overhangs. In one embodiment, an siRNA molecule has a two nucleotide 3′ overhang. In one embodiment, an siRNA is 21 nucleotides in length with two nucleotide 3′ overhangs (i.e. they contain a 19 nucleotide complementary region between the sense and antisense strands). In certain embodiments, the overhangs are UU or dTdT 3′ overhangs. Generally, siRNA molecules are completely complementary to one strand of a target DNA molecule, since even single base pair mismatches have been shown to reduce silencing. In other embodiments, siRNAs may have a modified backbone composition, such as, for example, 2′-deoxy- or 2′-O-methyl modifications. However, in preferred embodiments, the entire strand of the siRNA is not made with either 2′ deoxy or 2′-O-modified bases.

In one embodiment, siRNA target sites are selected by scanning the target mRNA transcript sequence for the occurrence of AA dinucleotide sequences. Each AA dinucleotide sequence in combination with the 3′ adjacent approximately 19 nucleotides are potential siRNA target sites. In one embodiment, siRNA target sites are preferentially not located within the 5′ and 3′ untranslated regions (UTRs) or regions near the start codon (within approximately 75 bases), since proteins that bind regulatory regions may interfere with the binding of the siRNP endonuclease complex (Elshabir, S. et al. Nature 411:494-498 (2001); Elshabir, S. et al. EMBO J. 20:6877-6888 (2001)). In addition, potential target sites may be compared to an appropriate genome database, such as BLAST, available on the NCBI server at www.ncbi.nlm, and potential target sequences with significant homology to other coding sequences eliminated.

Short hairpin RNAs may also be used to inhibit or knockdown gene or nucleic acid expression according to the invention. Short Hairpin RNA (shRNA) is a form of hairpin RNA capable of sequence-specifically reducing expression of a target gene. Short hairpin RNAs may offer an advantage over siRNAs in suppressing gene expression, as they are generally more stable and less susceptible to degradation in the cellular environment. It has been established that such short hairpin RNA-mediated gene silencing (also termed SHAGging) works in a variety of normal and cancer cell lines, and in mammalian cells, including mouse and human cells. Paddison, P. et al., GENES DEV. 16(8):948-58 (2002). Furthermore, transgenic cell lines bearing chromosomal genes that code for engineered shRNAs have been generated. These cells are able to constitutively synthesize shRNAs, thereby facilitating long-lasting or constitutive gene silencing that may be passed on to progeny cells. Paddison, P. et al., PROC. NATL. ACAD. Sci. USA 99(3):1443-1448 (2002).

ShRNAs contain a stem loop structure. In certain embodiments, they may contain variable stem lengths, typically from 19 to 29 nucleotides in length, or any number in between. In certain embodiments, hairpins contain 19 to 21 nucleotide stems, while in other embodiments, hairpins contain 27 to 29 nucleotide stems. In certain embodiments, loop size is between 4 to 23 nucleotides in length, although the loop size may be larger than 23 nucleotides without significantly affecting silencing activity. ShRNA molecules may contain mismatches, for example G-U mismatches between the two strands of the shRNA stem without decreasing potency. In fact, in certain embodiments, shRNAs are designed to include one or several G-U pairings in the hairpin stem to stabilize hairpins during propagation in bacteria, for example. However, complementarity between the portion of the stem that binds to the target mRNA (antisense strand) and the mRNA is typically required, and even a single base pair mismatch is this region may abolish silencing. 5′ and 3′ overhangs are not required, since they do not appear to be critical for shRNA function, although they may be present (Paddison et al. (2002) GENES & DEV. 16(8):948-58).

c. Mutation and Dominant Negative Inhibitors

The activity of a molecule may be altered by a variety of methods known and available in the art, in addition to knockout and knockdown of gene expression. For example, in certain embodiments, the activity of a molecule is disrupted by mutating a gene encoding the molecule. In other embodiments, the activity of a molecule is altered by overexpression of the molecule or a molecule that it cooperates with, such as, e.g., a binding partner. In yet another embodiment, the activity of a molecule is altered by expressing a dominant negative inhibitor of the molecule.

In one embodiment, the activity of a molecule is altered by expressing a dominant negative in the form of a pseudosubstrate inhibitor. For example, the ˜90 amino acid poxyiral proteins show striking similarity to the N-terminal third of eIF2 alpha; however, the viral proteins lack a phosphorylatable residue in the position analogous to Ser-51 in eIF2 alPHA. It has been demonstrated that the poxyiral proteins are pseudosubstrate inhibitors of PKR. Expression of either the K3L or C8L protein reduced eIF2 alpha phosphorylation and blocked the toxic effects associated with expression of PKR in yeast. This inhibition of PKR by the K3L and C8L proteins is dependent on a sequence motif (KGYID) conserved among all K3L homologs and eIF2 alpha and located roughly 30 residues C-terminal of the Ser-51 phosphorylation site in eIF2 alpha. K3L and C8L proteins inhibit PKR expressed in mammalian cells in a manner dependent on the critical residues required for their anti-PKR functions in yeast (Kawagishi-Kobayashi et al, 1997; 2000). These studies indicate that PKR recognition of K3L and eIF2a is likely to involve sequence elements remote from the Ser-51 phosphorylation site. Accordingly, in one embodiment, the activity of PKR may be altered by expression of all or a portion of a K3L or C8L protein. Similarly, the activity of eIF2 alpha may be increased by overexpression of eIF2 alpha or by overexpression of a region of eIF2 alpha comprising the PKR phosphorylation site, yet inactive in eIF2 alpha's normal function in protein expression. Without being bound by theory, this overexpressed region of eIF2 alpha binds cellular PKR, thus reducing its phosphorylation of endogenous eIF2 alpha.

In another embodiment, the activity of a molecule is altered by expressing a dominant negative mutant inhibitor of RNase L, as described in Castelli, J. et al., BIOMED PHARMACOTHER, (1998), 52:386-90). It has been shown that expression of this dominant negative mutant inhibited the activity of RNase L and suppressed poly(1)Ypoly(c)-induced apoptosis in interferon-primed fibroblasts. In addition, this dominant negative mutant inhibited poliovirus-induced apoptosis in HeLa cells.

In yet another embodiment, the activity of a molecule is inhibited by expressing a mutant or dominant negative form of PKR-activating protein (PACT). PKR is a recently identified cellular protein capable of activating PKR, as described in D'Acquisto, F. and Ghosh, S., SCI STKE (2001), 89:RE1).

In yet other embodiments, the invention contemplates altering the activity of a molecule by expressing a dominant negative mutant of PKR. A wide range of effective mutants of PKR may be used according to the invention. In certain embodiments, a PKR dominant negative mutant is capable of binding RNase L but is not capable of phorphorylating RNase L. For example, a PKR dominant negative mutant may be mutated at a residue critical for kinase activity. In one embodiment, a PKR mutant has reduced or no kinase activity.

d. Expression Constructs

As described supra, the activity of a molecule associated with nonspecific gene suppression may be altered by a variety of means, some of which employ the use of expression constructs, alone or, for example, in combination with knockout or knockdown vectors and reagents. Furthermore, knockdown reagents may be expressed in a cell using expression constructs. In certain embodiments, expression constructs are transiently present in a cell, while in other embodiments, they are stably integrated into a cellular genome. Furthermore, it is understood that due to the inherent degeneracy of the genetic code, other DNA sequences that encode substantially the same or a functionally equivalent amino acid sequence may be produced and these sequences may be used to express a given polypeptide.

Methods well known to those skilled in the art may be used to construct expression vectors containing sequences encoding a polynucleotide or polypeptide of interest and appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y. In one embodiment, expression constructs of the invention comprise polynucleotide sequences encoding all or a region of a molecule associated with nonspecific gene suppression in addition to regulatory sequences that govern expression of coding sequences.

Regulatory sequences present in an expression vector include those non-translated regions of the vector, e.g., enhancers, promoters, 5′ and 3′ untranslated regions, which interact with host cellular proteins to carry out transcription and translation. Such elements may vary in their strength and specificity. Depending on the vector system and cell utilized, any number of suitable transcription and translation elements, including constitutive and inducible promoters, may be used.

In mammalian cells, promoters from mammalian genes or from mammalian viruses are generally preferred, and a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T. (1984) PROC. NATL. ACAD. SCI. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. Exogenous translational elements and initiation codons may be of various origins, both natural and synthetic. The efficiency of expression may be enhanced by the inclusion of enhancers which are appropriate for the particular cell used, such as those described in the literature (Scharf, D. et al. (1994) RESULTS PROBL. CELL DIFFER. 20:125-162).

It is understood that constitutively decreasing or reducing expression of a component of nonspecific gene suppression may have deleterious effects on a cell. In addition, constitutive knockout or knockdown of a target gene may also have deleterious effects on a cell. Thus, in certain embodiments, the invention provides for the conditional expression or conditional knockout or knockdown of molecules associated with nonspecific gene suppression, or fragments, mutants or variants thereof. For example, an expression construct that expresses a knockdown reagent may comprise a regulatable element that permits conditional expression of the knockdown reagent and, thus, conditional knockdown of the molecule. In addition, a conditional expression construct may be used to drive expression of a molecule encoded by a gene that is knocked out in a cell, thus permitting the conditional knockout of the encoded molecule. Methods of conditional knockout and knockdown previously described are included within the scope of the present invention. These methods and reagents include, for example, those described in U.S. patent application Ser. Nos. 10/441,923 and 10/291,235, which are incorporated by reference in their entirety.

A variety of conditional expression systems are known and available in the art for use in both cells and animals, and the invention contemplates the use of any such conditional expression system to regulate the expression of a knockdown reagent. In certain embodiments of the invention, the use of prokaryotic repressor or activator proteins is advantageous due to their specificity for a corresponding prokaryotic sequence not normally found in a eukaryotic cell. One example of this type of inducible system is the tetracycline-regulated inducible promoter system, of which various useful version have been described (See, e.g. Shockett and Schatz, PROC. NATL. ACAD. SCI. USA 93:5173-76 (1996) for a review). In one embodiment of the invention, for example, expression of a molecule can be placed under control of the REV-TET system. Components of this system and methods of using the system to control the expression of a gene are well-documented in the literature, and vectors expressing the tetracycline-controlled transactivator (tTA) or the reverse tTA (rtTA) are commercially available (e.g. pTet-Off, pTet-On and ptTA-2/3/4 vectors, Clontech, Palo Alto, Calif.). Such systems are described, for example, in U.S. Pat. No. 5,650,298, U.S. Pat. No. 6,271,348, U.S. Pat. No. 5,922,927, and related patents, which are incorporated by reference in their entirety.

Briefly, in certain embodiments, these vectors express fusion proteins of the VP16 transactivator (tTA or rtTA) that activate transcription in the absence or presence of doxycycline, respectively. Thus, in certain embodiments, the presence of doxycycline or tetracycline prevents expression of an inhibitory regulatory molecule. In other embodiments, the presence of doxycycline or tetracycline permits expression of an inhibitory regulatory molecule. For example, expression of an antisense RNA, ribozyme, or RNAi molecule may be placed under control of a VP16 responsive promoter, and their expression regulated by the addition of doxycycline to media. Once activated, the transcribed molecules are free to associate with the target protein mRNA, leading to degradation of the mRNA. Specific REV-TET systems are described in Gossen, M. and Bujard, H. (1992) PROC NATL ACAD SCI USA 89, 5547-51 and Baron, U., Schnappinger, D., Helbl, V., Gossen, M., Hillen, W. and Bujard, H. (1999) PROC NATL ACAD SCI USA 96,1013-1018, and references cited within.

It should be understood that the present invention allows for considerable flexibility and a wide range of suitable inducible promoter and corresponding inducing agents, when used. In some embodiments of the invention, the choice of an inducible promoter may be governed by the suitability of the required inducing agent. Factors such as cytotoxicity or indirect effects on nontarget genes may be important to consider. In other instances, the choice may be governed by the properties of the inducible system as a whole. Examples of factors that might be important to consider include the ease with which the system can be introduced into the appropriate cell and the speed and strength with which induction of the system occurs following exposure to an inducing agent. Again, it is reiterated that the particular system chosen to induce or activate an effector of repression through a regulatable gene expression inhibitor sequence may operate in the presence of absence of an inducing agent, depending on the particular system chosen. Thus, in certain embodiments, cells will be maintained in an agent or compound to avoid repression of the disrupted gene, while in other embodiments, an agent or compound will be added to induce repression of a disrupted gene.

In certain embodiments, including, e.g., transgenic animals, polypeptides are expressed under the control of a tissue-specific or stage-specific regulatory element, which directs expression of an operably linked polynucleotide in a tissue- or developmental stage-specific manner. A variety of tissue- and stage-specific promoter and enhancer sequences are known and available in the art, which may be used according to the present invention.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul. Minn.) and Maddox, D. E. et al. (1983; J. Exp. Med. 158:1211-1216).

3. Applications

Methods and compositions of the invention related to reducing the activity of a molecule associated with nonspecific gene suppression have a variety of uses. For example, such methods and compositions may be used to establish cell lines and animals having constitutive or inducible reduction in nonspecific gene suppression, as described further infra. Such cells and animals may be used in a variety of ways to identify genes associated with or even required for any biological activity or process. Furthermore, the methods and compositions of the invention permit the use of long RNAi molecules, which offer increased efficacy and require less screening than short RNAi molecules.

B. Methods and Compositions for Performing RNA Interference

In one aspect, the present invention provides methods and compositions for performing RNAi. In certain embodiments, these methods and compositions are directed to the use of long RNAi molecules, including those previously shown to induce nonspecific gene suppression in mammalian cells. These methods and compositions may be performed or used in the presence of an agent that alters the activity of a molecule associated with nonspecific gene suppression or they may be performed or used in the absence of an agent that alters the activity of a molecule associated with nonspecific gene suppression.

The inventive methods and compositions directed to the use of long RNAi reagents offer several advantages over conventional RNAi methods and compositions directed to short RNAi molecules. In particular, long RNAi reagents are cleaved within the cell into shorter pieces, so the use of a single long RNA reagent is similar to using multiple short RNAi reagents. Accordingly, it is not necessary to screen multiple short RNAi reagents to identify one that mediates RNAi. For example, if the long RNAi reagent corresponds to all or substantially all of a gene or coding region of a gene, then presumably short RNAi reagents corresponding to all or substantially all of the gene or coding region of the gene will be generated within the cell to which the long RNAi agent is introduced, including at least some short RNAi reagents that are effective in mediating RNAi.

1. RNAi Methods

In one embodiment, the invention provides a method of performing RNAi, comprising introducing into a cell both an RNAi reagent, e.g., a long RNAi reagent, and an agent that reduces nonspecific gene suppression in response to long RNAi reagents. According to this embodiment of the invention, the reduction in nonspecific gene suppression caused by the agent facilitates the use of a long RNAi reagent.

In a related embodiment, the invention provides a method of performing RNAi, comprising introducing into a cell a long dsRNAi agent. In certain embodiments, a marker gene is also introduced into the cell. According to this embodiment of the invention, effects of the RNAi reagent on expression of the marker gene may be detected even in the presence of any nonspecific gene suppression caused by the long dsRNAi reagent.

The RNAi methods of the invention may be performed using any of the RNAi reagents described herein, including both double-stranded RNAi reagents and expression vectors capable of expressing RNAi reagents. In addition, the RNAi methods of the invention may be performed in any cell or animal described herein, including mammalian cells and mammals.

In certain embodiments, an RNAi method or composition of the invention causes a reduction in expression of a targeted gene. The reduction of expression may be greater than 10%, 33%, 50%, 75%, 90%, 95%, 99% or 100% as compared to a cell not treated according to the invention, as measured at any time point following introduction of the RNAi reagent to a cell, such as, e.g., 24 h, 48 h., 72 h., 96 h., or 1 week. The reduction in expression of a target gene may be readily determined by methods widely known and available in the art, including, e.g., PCR and northern blot analysis using primers or probes specific for the target gene.

2. Long RNAi Reagents

RNAi reagents of this aspect of the invention include all of the various types of RNAi reagents described supra, including RNA:RNA hybrids, DNA sense:RNA antisense hybrids, RNA sense:DNA antisense hybrids and DNA:DNA hybrids. An RNAi reagent is a long RNAi reagent if it has a double-stranded region of at least 30 nucleotides in length. In certain embodiments, long RNAi molecules comprise a double-stranded region at least 30 nucleotides in length, at least 50 nucleotides in length, at least 100 nucleotides in length, at least 200 nucleotides in length, at least 500 nucleotides in length, or at least 1000 nucleotides in length. In other embodiments, long RNAi molecules comprise a double-stranded region between 30 and 1000 nucleotides in length, between 50 and 1000 nucleotides in length, between 100 and 1000 nucleotides in length, or between 100 and 500 nucleotides in length. In one embodiment, a long RNAi molecule comprises a double-stranded region corresponding to the sequence of a full length cDNA sequence. In other embodiments, a long RNAi molecule comprises a double-stranded region that includes sequence corresponding to both coding and noncoding regions of a gene. In certain embodiments, the noncoding regions are 5′ or 3′ regions located upstream or downstream of the coding sequence of a gene, respectively.

The double-stranded region of an RNAi molecule may be formed by a single self-complementary polynucleotide strand or to complementary polynucleotides. Double-strandedness or duplex formation may be initiated inside or outside of a cell. The RNAi molecule may be introduced in any amount that allows delivery of at least one copy per cell. Higher doses (e.g., at least 5, 10, 100, 500 or 1000 copies per cell) of double-stranded material may yield more effective inhibition; while lower doses may be sufficient for particular applications.

RNAi molecules may be synthesized either in vivo or in vitro. For example, double-stranded RNAi molecules may be prepared in vitro by chemically synthesizing two polynucleotides with complementary regions and annealing the two strands to each other. Alternatively, each stand may be expressed in vitro using a vector suitable for in vitro transcription and the two strands may be annealed. The strands may be expressed in the same or different in vitro transcription reactions. Vectors and kits for in vitro transcription are available in the art, including, e.g., the Silencer™ siRNA Construction Kit from Ambion (Auston, TX)

So far, injection and transfection of RNAi molecules into cells and organisms have been the main method of delivery of RNAi molecules, such as siRNA and shRNA. While the silencing effect lasts for several days and appears to be transferred to daughter cells, it does eventually diminish. Recently, however, a number of groups have developed expression vectors to continually express siRNAs in transiently and stably transfected mammalian cells (see, e.g., Brummelkamp T R, et al. (2002). SCIENCE 296:550-553). Some of these vectors have been engineered to express small hairpin RNAs (shRNAs), which get processed in vivo into siRNAs-like molecules capable of carrying out gene-specific silencing. In certain embodiments, the vectors contain the shRNA sequence between a polymerase III (pol 111) promoter and a 4-5 thymidine transcription termination site. The transcript is terminated at position 2 of the termination site (pol III transcripts naturally lack poly(A) tails) and then folds into a stem-loop structure with 3′ UU-overhangs. The ends of the shRNAs are processed in vivo, converting the shRNAs into ˜21 nt siRNA-like molecules, which in turn initiate RNAi.

Another siRNA expression vector developed by a different research group encodes the sense and antisense siRNA strands under control of separate pol III promoters (Miyagishi M, and Taira K. (2002). NATURE BIOTECHNOL. 20:497-500). The siRNA strands from this vector, like the shRNAs of the certain other vectors, have 5 thymidine termination signals. Silencing efficacy by both types of expression vectors was comparable to that induced by transiently transfecting siRNA.

RNAi molecules may be introduced into a cell by any means available, including, e.g., electroporation, injection, transfection, and scrape-loading. In certain embodiments, RNAi molecules are directly introduced into a cell, while in other embodiments, RNAi molecules are introduced into the media containing a cell and then taken up by the cell via a passive or active mechanism.

3. Targets

RNAi reagents of the invention may be used to target any gene within a cell, including, e.g., endogenous genes, transgenes, and genes of a pathogen in the cell. The present invention is not limited to any particular type of target gene or nucleotide sequence. However, exemplary classes of target genes include developmental genes (e.g., adhesion molecules, cyclin kinase inhibitors, Writ family members, Pax family members, Mad family members, Winged helix family members, Hox family members, nuclear hormone receptor family members, cytokines/lymphokines and their receptors, growth/differentiation factors and their receptors, neurotransmitters and their receptors), oncogenes (e.g., ABL1, BCL1, BCL2, BCL6, CBFA2, CBL, CSFIR, ERBA, ERBB, ERBB2, ETSI, ETS1, ETV6, FGR, FOS, FYN, HCR, HRAS, JUN, KRAS, LCK, LYN, MDM2, MLL, MYB, MYC, MYCLI, MYCN, NRAS, PIM1, PML, RET, SRC, TALI, TCL3 and YES); tumor suppressor genes (e.g., APC, BRCA1, BRCA2, MADH4, MCC, NF1, NF2, RB1, TP53 and WT1); enzymes (e.g., ACC synthases and oxidases, ACP desaturases and hydroxylases, ADP-glucose pyrophorylases, ATPases, alcohol dehydrogensases, amylases, amyloglucosidases, catalases, cellulases, chlcone synthases, GTPases, helicases, hemicellulases, integrases, inulinases, invertases, isomerases, kinases, lactases, lipases, lipoxygenases, lysozymes, nopaline synthases, octopine synthases, pecinesterases, peroxidases, phosphatases, phospholipases, phosphbrylases, phytases, plant growth regulator synthases, polygalacturonases, proteinases and peptidases, pullanases, recombinases, reverse transcriptases, RUBISCOs, topoisomerases, and xylanases); apoptosis genes (e.g., bcl genes, Ced-3, human ICE (interleukin-1-P converting enzyme) (caspase-1), ICH-1 (caspase-2), CPP32 (caspase-3), ICErelil (caspase-4), ICErelIll (caspase-5), Mch2 (caspase-6), ICE-LAP3 (caspase-7), Mch5 (caspase-8) ICE-LAP6 (caspase-9), Mch4 (caspase-10), caspases 11-14, and others).

The sequences within the RNAi agent that share homology with or are complementary to a target gene are referred to herein as target regions of the RNAi agent. Target regions of RNAi agents of the invention generally comprise a sequence corresponding to a eukaryotic or mammalian gene being targeted for inhibition, although the invention also contemplates RNAI agents containing target regions corresponding to any organism, including, e.g., plants, animals, protozoa, bacteria, viruses, and fungi. An animal may be a vertebrate or an invertebrate. The target region may share 100% identity to a gene, or it may be a variant of a gene sequence.

Generally, RNAi reagents comprising a target region identical to a portion of the targeted gene are preferred. However, target regions comprising insertions, deletions and mutations relative to a targeted gene have also been shown to be effective in inhibition. The invention, therefore, includes RNAi reagents comprising a target region having at least 75% identify, at least 90% identity, at least 95% identity, at least 98% identity, at least 99% identity, and 100% identity to a targeted gene. It is also recognized that long RNAi reagents tolerate greater sequence variation more readily than short RNAi reagents, since the long RNAi reagents is processed into multiple short regions, some of which have less sequence variation than the long dsRNAi reagent as compared to the targeted gene sequence. For example, certain long dsRNAi reagents are processed into small regions, one or more of which may have 100% identity to a targeted gene sequence and also be effective in mediating RNAi of the target gene.

C. Cells, Libraries, Arrays and Animals

In addition to methods and compositions related to RNAI, the invention also includes cell, libraries, arrays and animals comprising RNAi reagents or cells according to the invention. Cells, libraries, arrays and animals of the invention may include either or both of an RNAi reagent, e.g., a long RNAi reagent, and an agent that alters the activity of a molecule associated with nonspecific gene suppression.

1. Cells

The present invention includes cells comprising an agent that alters the activity of a molecule associated with nonspecific gene suppression. The agent may be transiently presenting the cell or stably integrated into a cellular genome. It is understood that such agents include both polynucleotides and polypeptides that alter activity, as well as polynucleotides capable of expressing a polynucleotide or polypeptide that alters activity. Specific agents have been described supra, and it is understood that a cell of the invention may comprise any of these agents.

In one embodiment, the invention includes a cell line wherein each or the majority of cells comprises an agent that alters the activity of a molecule associated with nonspecific gene suppression. Such a cell line is particularly useful, since it may be used in concert with any long RNAi molecule. In one embodiment, the cell line comprises a polynucleotide comprising a sequence that encodes an agent that alters the activity of a molecule associated with nonspecific gene suppression operably linked to a regulatable promoter, such as, e.g., an inducible promoter, wherein expression of the agent may be induced as desired. In certain embodiments, the polynucleotide may be stably integrated within the cellular genome; in another embodiment, the polynucleotide may be present in an episome or a replicating polynucleotide. In certain embodiment, the cell line comprises immortalized cells; in another embodiment, the cell line comprises transformed cells.

In another embodiment, a cell of the invention comprises a long RNAi molecule. The molecule may be transiently present in the cell or stably integrated into a cellular genome. It is understood that such agents include RNAi molecules, as well as polynucleotides capable of expressing an RNAi molecule. Specific RNAi molecules have been described supra, and it is understood that a cell of the invention may comprise any of these agents. It is further understood that the terms RNAi reagent and RNAi molecule are used interchangeably herein.

In an embodiment, the invention includes a cell line comprising a long RNAi molecule. In certain embodiments, the long dsRNAi molecule is expressed from a polynucleotide present in the cell. The RNAi molecule may be expressed constitutively or inducibly.

In certain embodiments, cells of the invention are primary cells, cell lines, immortalized cells, or transformed cells. A cell may be a somatic cell or a germ cell. The cell may be a non-dividing cell, such as a neuron, or it may be capable of proliferating in vitro in suitable cell culture conditions. Cells may be normal cells, or they may be diseased cells, including those containing a known genetic mutation. Eukaryotic cells of the invention include mammalian cells, such as, for example, a human cell, a murine cell, a rodent cell, and a primate cell. In one embodiment, a target cell of the invention is a stem cell, which includes, for example, an embryonic stem cell, such as a murine embryonic stem cell. In another embodiment, a target cell is a differentiated cell, such as, e.g., adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chrondrocytes, osteoblasts, osteoclasts, hepatocytes, and cells of the endocrine or exocrine glands.

Cells may contain polynucleotides integrated in one or more alleles of a disrupted gene. In certain embodiments, following selection, the target cell will contain disruptions in both or all alleles of a disrupted gene. Cells containing disruption of both or all alleles of a gene may be produced by sequentially disrupting each allele, or by utilizing a selection procedure that preferably selects for cells wherein both alleles are disrupted. For example, wherein a selectable marker confers resistance to a drug upon a target cell, cells containing disruption of both or all alleles may be selected by using an increased concentration of the drug. In one embodiment of the invention, a first allele of a gene is disrupted by insertion of a gene trap or targeting vector, and a second allele of the same gene is disrupted by subsequent insertion of a targeting vector. When the cassette comprising the selection marker of the first insertion vector is removed via excision using site-specific recombinases prior to disruption of a second allele, it is possible to re-use in the second insertion vector the same selection marker that was used in the first selection marker. Alternatively, a different selection marker may be used to disrupt different alleles, e.g. neo, hygro, puro, etc.

2. Libraries and Arrays

The invention further provides libraries and arrays of compositions of the invention. In one embodiment, a library or array comprises a plurality of cells comprising RNAi molecules of the invention. Cells of such a library or array may further comprise an agent capable of altering the activity of a molecule associated with nonspecific gene suppression. In one embodiment, the invention includes libraries and arrays of long RNAi agents according to the invention.

The cells of a library, collection or array may each comprise different disrupted or targeted genes or different RNAi molecules. The libraries and arrays may comprise pools of two or more cells or molecules or may comprise individually isolated cells or molecules. In addition, the libraries, arrays and collections may comprise multiple groups of vessels. In particular embodiments, a library or array comprises at least 10 different RNAi molecules, at least 100 different RNAi molecules, at least 500 different RNAi molecules, at least 1000 different RNAi molecules, at least 5000 different RNAi molecules, at least 10,000 different RNAi molecules, at least 25,000 different RNAi molecules, at least 50,000 different RNAi molecules, at least 100,000 different RNAi molecules, or at least 200,000 different RNAi molecules. In related embodiments, a library or array of the invention comprises RNAi molecules directed to at least two different genes, at least 10 different genes, at least 100 different genes, at least 500 different genes, at least 1000 different genes, at least 5000 different genes, at least 10,000, at least 20,000, at least 50,000, or at least 100,000 different genes. In one embodiment, a library or array of the invention comprises RNAi molecules directed to all or substantially all genes within a genome. In another embodiment, a library or array of the invention comprises a plurality of RNAi molecules that all target one gene or a plurality of genes within a particular biological pathway, such as, e.g., genes associated with the apoptotic caspase family or genes encoding Mad family members.

Arrays of the invention include both standard or large arrays and microarrays. Microarrays are miniaturized devices typically with dimensions in the micrometer to millimeter range and are particularly suited for embodiments of the invention. Microarrays are particularly desirable for their virtues of high sample throughput and low cost for generating data.

Arrays of the invention may comprise either cells or RNAi molecules. In certain embodiments, the RNAi molecules are long RNAi molecules. Accordingly, a variety of different types of array devices may be used. For example, an array may comprise a surface upon which samples may be placed in discrete locations. In addition, an array may comprise discrete wells capable of holding a fluid sample comprising cells or RNAi molecules. Locations of an array may be physically separated from each other by a barrier or they may be contiguous and lack any physical means of separation. In certain embodiments, an array comprises positionally addressable locations, such that it is possible to determine the molecule or cell located at one or more locations.

Arrays may be constructed via microelectronic and/or microfabrication using essentially any and all techniques known and available in the semiconductor industry and/or in the biochemistry industry, provided only that such techniques are amenable to and compatible with the deposition and screening of polynucleotide sequences. For example, the light-directed chemical synthesis process developed by Affymetrix (see, U.S. Pat. Nos. 5,445,934 and 5,856,174) may be used to synthesize biomolecules on chip surfaces by combining solid-phase photochemical synthesis with photolithographic fabrication techniques. The chemical deposition approach developed by Incyte Pharmaceutical uses pre-synthesized polynucleotides for directed deposition onto chip surfaces (see, e.g., U.S. Pat. No. 5,874,554). Other useful technology that may be employed is the contact-print method developed by Stanford University, which uses high-speed, high-precision robot-arms to move and control a liquid-dispensing head for directed polynucleotide deposition and printing onto chip surfaces (see, Schena, M. et al. SCIENCE 270:467-70 (1995)). The University of Washington at Seattle has developed a single-nucleotide probe synthesis method using four piezoelectric deposition heads, which are loaded separately with four types of nucleotide molecules to achieve required deposition of nucleotides and simultaneous synthesis on chip surfaces (see, Blanchard, A. P. et al., BIOSENSORS & BIOELECTRONICS 11:687-90 (1996)).

Further examples of technology contemplated for use in making and using arrays are provided in “Genome-wide expression monitoring in Saccharomyces cerevisiae.” by Wodicka, L. et al. (Nature Biotechnol. 15:1359-1367 (1997)), “Genomics and Human disease—variations on variation.” by Brown, P. O. and Hartwell, L. and “Towards Arabidopsis genome analysis: monitoring expression profiles of 1400 genes using cDNA microarrays.” by Ruan, Y. et al. (The Plant Journal 15:821-833 (1998)). Additional microarray technologies that may be utilized according to the present invention include, for example, electronic microarrays, including, e.g. the NanoChip Electronic Microarray, which is available from Nanogen, Inc. (San Diego, Calif.) and described in detail in U.S. Pat. No. 6,258,606, “Multiplexed Active Biologic Array”; U.S. Pat. No. 6,287,517, “Laminated Assembly for Active Bioelectronic Devices”; U.S. Pat. No. 6,284,117, “Apparatus and Method for Removing Small Molecules and Ions from Low Volume Biological Samples”; U.S. Pat. No. 6,280,590, “Channel-Less Separation of Bioparticles on a Bioelectronic Chip by Dielectrophoresis”; and U.S. Pat. No. 6,254,827, “Methods for Fabricating Multi-Component Devices for Molecular Biological Analysis and Diagnostics, and references cited therein, all of which are incorporated by reference in their entirety.

3. Animals

The invention also includes animals comprising a polynucleotide or cell of the invention. Animals according to the invention are typically nonhuman animals. In certain embodiments, the animals are mammals, such as, for example, nonhuman primates (e.g., monkeys), mice, rats, dogs, or cats. In particular embodiments, animals include animal models of disease. In certain embodiments, animals of the invention are knockout animals wherein one or more alleles of a gene are disrupted. In another embodiment, animals are transgenic animals. Transgenic animals of the invention may comprise any of a variety of introduced polynucleotides, including sequences that express a knockdown reagent or sequences encoding an agent that alters the activity of a molecule associated with nonspecific gene suppression.

In certain embodiments, animals of the invention express a transgene in a tissue-specific or developmentally-specific manner. In other related embodiments, a transgene is expressed in an inducible manner. Similarly, the invention includes cells and animals with conditional knockout of one or more alleles of a gene.

Methods for obtaining transgenic and knockout animals are well known in the art. Methods of generating a mouse containing an introduced gene disruption are described, for example, in Hogan, B. et al., (1994), MANIPULATING THE MOUSE EMBRYO: A LABORATORY MANUAL, 2nd ed., Cold Spring Harbor Laboratory Press, Cold Spring Harbor, N.Y. and Joyner. In one embodiment, gene targeting, which is a method of using homologous recombination to modify a cell's or animal's genome, can be used to introduce changes into cultured embryonic stem cells. By targeting a target gene of interest in ES cells, these changes can be introduced into the germlines of animals to generate chimeras and knock-out animals.

Generally, the ES cells used to produce the knockout animals will be of the same species as the knockout animal to be generated. Thus, for example, mouse embryonic stem cells are used for generation of knockout mice. Embryonic stem cells are generated and maintained using methods well known in the art such as those described, for example, in Doetschman, T., et al., J. Embryol. Exp. Morphol. 87:27-45 (1985), and improvements thereof. Any line of ES cells may be used according to the invention. However, the line chosen is typically selected for the ability of the cells to integrate into and become part of the germ line of a developing mouse embryo so as to create germ line transmission of the knockout construct. One example of a mouse strain commonly used for production of ES cells is the 129J strain. Other examples include the murine cell line D3 (American Type Culture Collection, catalog no. CKL 1934) and the WW6 cell line (Ioffe, et al., PNAS 92:7357-7361). The cells are cultured and prepared for knockout construct insertion using methods well known to one of ordinary skill in the art, such as those set forth by Robertson in: TERATOCARCINOMAS AND EMBRYONIC STEM CELLS: A PRACTICAL APPROACH, E. J. Robertson, ed. IRL Press, Washington, D.C. (1987); by Bradley et al., CURRENT TOPICS IN DEVEL. BIOL. 20:357-371 (1986); and by Hogan et al., MANIPULATING THE MOUSE EMBRYO: A LABORATORY MANUAL, Cold Spring Harbor Laboratory Press, Cold Spring Harbor, N.Y. (1986). Other preferred methods of the invention include those methods described in PCT application WO/0042174 and PCT application WO/0051424, including double nuclear transfer.

4. Marker Genes

In a variety of embodiments, cells and animals of the invention comprise a marker gene. For example, in one embodiment, cells comprising a knockout of one or more alleles of a gene encoding a molecule associated with nonspecific gene suppression will also contain a marker gene. Marker genes are typically included in knockout constructs, so that cells that have integrated knockout vector sequences can be readily identified. In a different embodiment, cells of the invention may comprise a polynucleotide comprising a marker gene regulated by operably linked sequences, such as promoter and or enhancer or repressor sequences, in addition to an RNAi molecule. Such cells are useful, e.g., in determining the effect of a long dsRNAi molecule on expression of the marker gene.

Marker genes include reporters, positive selection markers, and negative selection markers. A reporter is any molecule, including polypeptides as well as polynucleotides, expression of which in a cell produces a detectable signal, such as luminescence, for example. A selection molecule is any molecule, including polypeptides as well as polynucleotides, expression of which allows cells containing the gene to be identified, such as antibiotic resistance genes and fluorescent molecules, for example. A negative selection marker is any molecule, including polypeptides and polynucleotides, expression of which inhibits cells containing the gene to be identified, such as the HSV-tk gene, for example. Exemplary markers are disclosed in U.S. Pat. No. 5,464,764 and No. 5,625,048, which are incorporated by reference in their entirety. Procedure for selecting and detecting markers are widely available and published in the art, including, for example, in Joyner, A. L., GENE TARGETING: A PRACTICAL APPROACH, 2nd ed., (2000), Oxford University Press, New York, N.Y.

Examples of reporter genes widely used in detecting the presence of a introduced polynucleotide include the E. coli β-galactosidase gene (lacZ), which is detected using an enzymatic assay with X-gal, the human placental alkaline phosphatase gene (HPAP), which is detected by an enzymatic assay using a substrate such as BM Purple AP Substrate (Boehringer Mannheim), and green fluorescent protein (GFP), and variants thereof (e.g. EGFP (Clontech Inc.), EYFP, and ECFP), which can be detected microscopically or by fluorescence activated cell sorting (FACS). In addition, glucose phosphate isomerase (GPI) may be used as a marker to detect chimeras by GPI cellulose-acetate electrophoresis.

A variety of different selection/selectable marker genes are available in the art to identify vector integration into genomic DNA. Selectable markers that may be used according to the invention, include, for example, dominant and negative section markers, as well as positive and negative selection markers. Examples of preferred selectable markers include neomycin phosphotransferase (neo), histidinol dehydrogenase (hisD), hygromycin resistance (hygro), thymidine kinase, blasticidin S deaminase (bsr) and puromycin-N-acetyltransferase (puro). Exemplary markers also include chloramphenicol-acetyl transferase (CAT), dihydrofolate reductase (DHFR), and β-galactosyltransferase. For a list of other mammalian selection markers, see Sambrook, J., et al., MOLECULAR CLONING: A LABORATORY MANUAL, 2nd ed. (2001), Cold Spring Harbor Laboratory Press, Cold Spring Harbor, N.Y. Methods of detecting a suitable selectable marker are available in the art and depend, in part, on the origin of the targeted cell.

Other appropriate selectable marker genes include fusion proteins comprising reporter and selector proteins, particularly in-frame fusions between lacZ and selector genes, such as the βgeo marker. For example, the marker comprising an in-frame fusion of lacZ and neo (βgeo) permits direct selection of vector integrations into expressed genes, since G418 resistant cells will only be obtained when the integration leads to the generation of a functional lacZ/neo or neo/lacZ fusion protein. Furthermore, selectable markers include cell surface proteins, including cell adhesion molecules, such as integrins.

In certain embodiments, a cell may contain a negative selection marker, such as those disclosed in U.S. Pat. No. 5,464,764, hereby incorporated by reference in its entirety. Negative selection methods typically involve removing cells that express the negative selection marker by, for example, killing them, sorting them based on fluorescence, or removing them by panning. Examples of negative selection markers that may be used according to the invention include xanthine/guanine phosphoribosyl transferase (gpt), herpes simplex thymidine kinase (HSVtk), and diphtheria toxin A fragment (DTA). For example, hypoxanthine-guanine phosphoribosyltransferase (Hprt) may be used in combination with Hprt-defective cells, while the bacterial guanine/xanthine phosphoribosyltransferase (gpt) permits growth on MAX medium (Song, K. Y., et al. (1987) PROC. NAT'L ACAD. SCI. U.S.A. 84, 6820-6824). When included within a vector of the invention, negative selection markers are generally included in addition to a reporter or positive selection marker.

Cells containing introduced or exogenous polynucleotides or vector sequences integrated within the cellular genome are selected by any means available in the art for the particular selection marker. For example, selection may be accomplished by growing cells transfected with a vector containing a positive selection marker in selective media that permits cell growth only when the positive selection marker is expressed, or by sorting cells based on marker expression, such as by expression of a florescent marker. Integration events may also be identified and confirmed by routine molecular biology techniques, including northern blotting and southern blotting. The identity of a gene disrupted by vector sequence insertion may be determined by sequencing genomic DNA surrounding the inserted vector sequence and aligning with the human genome database. Methods of obtaining genomic DNA and DNA sequencing are routine and known in the art.

In certain embodiments, endogenous promoters within genomic sequences drive marker gene expression. Alternatively, an exogenous promoter capable of driving marker gene expression may be included within the vector. In some instances, it may be preferable to include an exogenous promoter capable of driving marker gene selection to ensure that the marker gene is expressed at levels adequate for detection or selection. Promoters that may be used to drive expression of a marker gene are widely known and available in the art and include, for example, both mammalian and viral promoters, such as thymidine kinase promoters and cytomegalovirus promoters. In other instances, it may be preferable to include an exogenous promoter that normally drives expression of endogenous genes so that the effect of exogenous stimuli or agents on said promoter can be monitored through use of the marker gene.

5. Screening Arrays and Libraries of RNAi Reagents

In certain embodiments, the invention provides methods and reagents for screening RNAi reagents, including, e.g., long RNAi reagents, for their ability to alter the expression of a reporter gene. Such methods have a variety of useful applications, and are particularly valuable in identifying genes involved in a particular biological pathway of interest. Generally, these methods involve introducing a library of siRNA reagents (or vectors for producing these, etc.), including RNAi reagents to cells comprising a reporter gene, such that one or more RNAi reagents will be introduced or expressed into a plurality of cells. The cells are then screened by determining reporter gene expression according to any suitable means available in the art, and cells having altered levels of reporter gene expression, e.g., as compared to control cells that do not contain a RNAi reagent, are identified. The RNAi reagent present in the identified cell(s) is then identified. Altered levels of reporter gene expression include both increased and decreased levels of expression, including increases or decreases of at least 10%, 20%, 25%, 30%, 40%, 50%, 60%, 70%, 75%, 80%, 90%, 95%, or 100%.

In certain embodiments, such methods may be used to identify a gene that regulates a promoter governing expression of a reporter gene. According to one embodiment, a reporter gene is integrated into the genome of a cell, e.g., a mammalian cell, such that expression of the reporter gene is regulated by endogenous genomic sequences, including promoter and/or enhancer or repressor sequences. Cells comprising the integrated reporter are propagated and used to screen a library of RNAi reagents to identify an RNAi reagent that alters expression of the reporter gene, thus indicating that this RNAi reagent regulates the endogenous promoter and/or enhancer or repressor sequences governing expression of the reporter gene. In certain embodiment, the RNAi reagents are long RNAi reagents.

Reporter genes may be introduced into the genome of a cell by any of a wide variety of methods and using any of a wide variety of constructs available in the art. For example, a reporter gene may be introduced into the genome of a cell using gene trap or homologous recombination vectors, such as those used for to knockout an allele, including those described, e.g., in U.S. patent application Ser. Nos. 10/028,970, 10/291,235, and 10/441,923, which are incorporated by reference in their entirety. In one embodiment, a vector used to introduce a reporter gene into the genome of a cell comprises polynucleotide sequences of the reporter gene and an origin of replication. In another related embodiment, a vector used to introduce a reporter gene into the genome of a cell may comprise a reporter gene, an origin or replication, and one or more of the following sequences: a splice acceptor (e.g., human EF-1α intron 1 splice acceptor), an internal ribosomal entry site (IRES), a promoter operably linked to a marker gene, a polyadenylation signal (polyA) and one or more sequence-specific recombinase binding sites (e.g., loxP sites). Optionally, the vector may comprise a splice donor instead of or in addition to the polyA sequence. The vector may further comprise a bacterial selection marker.

In other embodiments, the reporter gene may be present in a vector that is not integrated into the genome of a cell. For example, the vector may be a plasmid transiently present in a cell or an episome that is stably introduced into a cell but not integrated into the cellular genome. In certain embodiments, the vector includes polynucleotide sequences comprising any combination of regulatory sequences, such as promoter, enhancer, and repressor/suppressor elements, including those derived from genomic sequences, those associated with a specific gene of interest, and/or any other regulatory sequences. Here, as in any vector or polynucleotide described herein, the regulatory sequences may be operably linked to the reporter gene, such that expression of the reporter gene is regulated by the regulatory sequences. In one embodiment, the vector is a recombination vector, such as those described in U.S. patent application Ser. Nos. 10/028,970 and 10/291,235. Accordingly, the reporter gene may be present in a vector that was prepared by random insertion of polynucleotide sequences comprising a reporter into a genome, followed by excision of the inserted sequences and surrounding flanking genomic sequence, and circularization of the excised sequences to produce a vector comprising a reporter gene having its expression regulated by the genomic sequences. The reporter gene may be the same one present in the original polynucleotide sequence inserted into the cellular genome, or it may be a different reporter gene, which was introduced following excision.

In certain embodiments, expression of the reporter gene is regulated by sequences known to be involved in a particular biological pathway of interest, e.g., cell proliferation, differentiation, apoptosis, senescence, or activation, or sequences known to regulate the expression of one or more genes. For example, in one embodiment, expression of the reporter gene is responsive to treatment of cells containing the reporter gene and its associated regulatory sequences, either integrated into the cellular genome or not, with a growth factor, such as, e.g., transforming growth factor β or epidermal growth factor. The regulatory sequences associated with reporter gene expression may be known sequences and may be sequences that regulate expression of an endogenous cellular gene, or they may be unknown sequences or sequences not known to be involved in regulating expression of a gene.

Accordingly, the invention includes methods and systems of identifying RNAi reagents and, thus, genes and their expressed polynucleotide and polypeptide products that are involved in any cellular process or that regulate any other gene. In one embodiment, these methods include the steps of: (1) randomly inserting polynucleotide sequences comprising a reporter gene into the genome of cells, e.g., mammalian cells, (2) identifying a cell wherein expression of the reporter gene is altered in response to a stimuli; (3) introducing a single RNAi reagent or a library of RNAi reagents, e.g., long RNAi reagents, into cells wherein expression of the reporter gene is altered in response to a stimuli; (4) identifying a cell having an introduced RNAi reagent where expression of the reporter gene is further altered as compared to in the absence of the particular RNAi reagent; and (5) determining the sequence of the RNAi reagent and, thereby, determining the identity of a gene whose expression is disrupted by the presence of the RNAi reagent, thereby determining the identify of a gene involved in regulating gene expression in response to the stimuli. The stimuli may be any of a wide range of treatments or conditions, including, but not limited to, alterations in temperature or CO₂, treatment with a growth factor or cytokine, inducement of proliferation, differentiation or apoptosis, and overexpression of a gene.

In other related embodiments, the gene of interest and, thus, the reporter gene, may be not regulated by a stimulus but may be associated with a biological or disease pathway. Such genes may be identified by any of a variety of methods, including, for example, using expression arrays, proteomics, bioinformatics, or other genomics methodologies. Similarly, the gene of interest may be a gene having a known toxicity, and pathways regulating its expression may be defined.

In one exemplary embodiment, polynucleotide sequences comprising a reporter gene are randomly inserted into different genomic sites in mammalian cells. These cells are then treated with a growth factor, and cells exhibiting altered levels of reporter gene expression in response to growth factor treatment are identified. These cells may then be isolated and propagated, and the genomic sequences regulating expression of the reporter gene may be identified. These cells are then used to screen a library of RNAi reagents, e.g., long RNAi reagents, to identify an RNAi reagent that causes altered expression of the reporter gene, in the presence or absence of growth factor treatment. In certain embodiments, the change of expression in response to growth factor treatment is compared between cells comprising RNAi reagents and control cells lacking an RNAi reagent. Cells displaying an altered change in reporter gene expression as compared to control cells, such as, e.g., cells not having an introduced RNAi reagent, are identified, the RNAi reagent is determined, and the corresponding gene that is disrupted by the RNAi reagent is identified as a gene involved in growth factor responsiveness.

In another related exemplary embodiment, an individual vector or a library of vectors derived from random insertion of a marker gene and subsequent excision of the marker gene and flanking genomic sequences (a recombination vector library) is prepared. Generally, a library of recombination vectors comprises two or more recombination vectors comprising different genomic polynucleotide sequences. In certain embodiments, the library comprises at least 10, at least 100, at least 1000, at least 5000, at least 10,000, at least 25,000, at least 50,000, at least 100,000, or at least 250,000 recombination vectors comprising different genomic polynucleotide sequences. The identity of the flanking genomic sequences is determined by any available method, such as, e.g., sequencing and alignment with the human genome database. The same marker gene used in the original insertion vector may be maintained in the recombination vector, or an alternative marker gene may be introduced into the recombination vector. These homologous recombination vectors are then used to introduce the marker gene into cells through homologous recombination, such that expression of the marker gene is directed by the prescribed transcriptional activity associated with the recombination site by, for example, placing expression of the marker gene under regulation of endogenous promoters/enhancers/repressors. These cells, which comprise marker genes integrated into their genome may be used to screen RNAi reagents, including, e.g., long RNAi reagents, for their ability to alter marker gene expression, thereby identifying the gene disrupted by the RNAi reagent as a gene involved in regulating expression of the gene normally regulated by the regulatory elements governing expression of the inserted marker gene.

In certain embodiments, one or more, e.g., a library of, recombination vectors is introduced into cells and screened to identify a cell wherein expression of the marker gene is altered in response to a stimulus or as compared to a control cell. For example, expression of a marker gene in a normal cell may be compared to expression of marker gene in a diseased cell to identify a cell having altered expression in the diseased cell. The identified cell may be used for screening RNAi reagents to identify genes.

In certain embodiments, such as, for example, when a recombination vector includes genomic sequences capable of regulating expression of the associated reporter gene, i.e., promoter and/or enhancer or repressor sequences, recombination vectors may be transiently introduced into cells for screening of RNAi reagents that effect expression of the reporter gene.

The skilled artisan will appreciate that the described methods have a wide variety of applications and may be used to RNAi reagents that increase or suppress expression of the reporter. Accordingly, these methods and reagents may be used to identify the pathway and genes implicated in regulation of expression of any particular gene. This, in turn, has implications and application to for pathway discovery, target identification and validation as well as identification of biomarkers (other genes that may be regulated by a pathway).

D. Applications

The invention further provides a variety of applications and uses of the inventive compositions and methods. As will be readily understood, the compositions and methods of the inventions can be used for performing RNAi according to any known or practiced method. The libraries and arrays of the invention offer particular advantages, since they can be used to identify genes, e.g., that alter a cellular characteristic, that alter the activity of a molecule, or that effect gene expression. In addition, cells, libraries and arrays of the invention may be used to test or screen drugs and drug candidates, including, but not limited to organic compounds, small organic molecules, gene therapy vectors, polynucleotide-based drugs and polypeptide-based drugs, for their ability to alter a cellular characteristic, alter the activity of a molecule, or effect gene expression in the presence of a composition of the invention.

In one embodiment, the invention provides methods that involve screening a library of RNAi reagents and identifying an RNAi reagent having a particular characteristic or effect on a cell trait, response or any other cellular property or characteristic. Without wishing to be bound by theory, it is believed that the RNAi reagent exerts its effect by reducing expression of a gene comprising the same polynucleotide sequence as at least a region of the RNAi reagent. The identity of the RNAi reagent and the gene targeted by the RNAi reagent may be determined by any means available in the art, including, e.g., by sequencing at least a portion of the RNAi reagent. The identity of the RNAi reagent may also be determined by other means, including, e.g., by its location or position in an array. In addition, RNAi reagents may be tagged in some manner, e.g, by the addition of a specific sequence or other indicator, so as to facilitate the identification of the RNAi reagent and corresponding gene.

In certain embodiments, the invention provides a method of determining a biological function of a gene using an RNAi molecule, e.g., a long RNAi molecule. According to one embodiment, an RNAi molecule is introduced into a cell. One or more biological traits, functions or characteristics of the cell are examined and compared to the same traits, functions or characteristics in a control cell, which lacks the introduced RNAi molecule. Differences between the two cells are indicative of the gene targeted by the RNAi molecule being associated with the altered trait, function or characteristic. Alternatively, a trait may be examined in a single cell before and after introduction of an RNAi molecule to similarly determine a function of the targeted gene. Accordingly, wherein the function of a gene is unknown, the invention provides a method of determining the function by introducing an RNAi molecule capable of reducing expression of the gene into a cell and identifying altered cellular characteristics, thereby identifying a function of the gene.

In another embodiment, the invention provides a method of determining the effect of reducing the expression of a gene on the expression of a marker gene. According to one embodiment, an RNAi molecule is introduced into a cell comprising a marker gene under the transcriptional control of an operably linked sequence, and the level of expression of the marker gene is determined and compared to the level of expression of the same marker gene in a cell that does not contain the introduced RNAi molecule. In certain embodiments, the RNAi molecule is a long RNAi molecule.

In a related embodiment, a library or array of cells comprising a plurality of RNAi molecules may be screened to identify a gene that regulates expression of a marker gene by introducing a marker gene into cells of such a library or array, determining expression of the marker gene in the cells, and identifying a cell wherein expression of the marker gene is altered as compared to a control cell that does not contain an RNAi molecule or other cells of the library or array. In one embodiment, this method may be employed to identify genes that regulate the expression of another gene. Regulatory sequences of a gene of interest may be operatively linked to a marker gene, so that these regulatory sequences regulate expression of the marker gene, and RNAi reagents that alter the expression of the marker gene are identified by screening a library of RNAi reagents, as described herein. Without wishing to be bound by theory, it is believed that the gene disrupted by the identified RNAi reagent is involved, either upstream or downstream, in regulating expression of the gene of interest. In certain embodiments, reduction in expression of a gene or reporter gene may be at least 10%, at least 25%, at 50%, at least 75%, at least 90%, at least 95%, at least 99%, or 100%.

The invention may also be used more generally to identify a gene associated with any particular biological attribute or characteristic. In one embodiment of such a method, an array of cells comprising a plurality of RNAi molecules of the invention is screened, and a cell having an altered biological attribute as compared to a control cell is identified. At least a portion of the sequence of the RNAi molecule present in the identified cell is determined and the corresponding gene is identified as a gene associated with the biological attribute. In certain embodiments, the RNAi molecules are long RNAi molecules.

In one particular embodiment, the invention includes a method of identifying a gene associated with growth or viability of tumor cells, by providing a library or array of tumor cells comprising a plurality of RNAi molecules and identifying a cell within the library or array having altered growth or viability as compared to other cells or to a control cell that does not comprise an RNAi molecule.

The invention also provides methods of identifying a gene associated with tumor cell sensitivity to a chemical agent, comprising providing a library or array of tumor cells comprising a plurality of RNAi molecules, treating the cells with a chemical agent, and identifying a cell having altered sensitivity to the chemical agent as compared to other cells or to a control cell. In one embodiment, following the identification of a cell, the gene is then identified by determining the sequence of at least a portion of the RNAi molecule and identifying the gene having expression disrupted by the RNAi molecule, based upon sequence homology. As with all methods of the invention, the RNAi molecules may be long RNAi molecules.

The methods and compositions of the invention also have a wide range of useful applications related to the identification or validation of drugs and drug candidates. For example, in one embodiment, the invention includes a method of identifying a gene that enhances or suppresses the effectiveness of a drug. Cells comprising a marker gene having its expression altered by exposure of the cells to a drug may be used to screen a library of RNAi reagents, e.g., long RNAi reagents, in the presence or absence of the drug. Cells displaying enhanced or reduced alterations in marker gene expression in response to the drug are identified. The RNAi reagent present in the cell and the gene disrupted by the RNAi reagent are identified, thereby identifying a gene that enhances or suppresses the effectiveness of a drug.

In a related embodiment, the invention includes methods of identifying targets of a gene with an unknown mechanism of action. This gene may be of interest for a variety of reasons. For example, it may have been identified as a gene having altered expression in response to treatment of cells with a drug or other agent, such as a growth factor or cytokine. According to the invention, cells comprising a marker gene having its expression regulated by regulatory sequences of the gene with an unknown mechanism of action is generated are used to screen a library of RNAi reagents, e.g., long RNAi reagents, to identify an RNAi reagent that alters expression of the marker gene. The gene targeted by the RNAi reagent is thus identified as a gene involved in the pathway that regulates the gene of unknown mechanism of action.

Such methods of identifying targets of a gene with an unknown mechanism of action may further be performed in conjunction with other procedures, including, for example, procedures to identify a compound that alters gene expression. For example, screens of RNAi reagents and screens of drug candidates may be performed to identify drugs and RNAi reagents that alter the expression of a marker gene regulated by genomic regulatory sequences. For example, in one embodiment, a gene with an unknown pathway may be implicated in disease by array, proteomics, bioinformatics, or any other method (including identification of a structure motif). A marker gene is operatively linked to the gene or regulatory sequences thereof, e.g., in a vector, by gene trapping, or by homologous recombination, and used to screen RNAi for modulators of expression. RNAi reagents that cause altered expression of the marker gene and the genes disrupted by these RNAi reagents are thus identified as regulatory genes in the pathway for the gene of interest. These genes may, therefore, be drug discovery targets, biomarkers, etc.

Targets of drug products or chemical entities with unknown mechanisms of action may be identified according to certain embodiments of the invention. Genes whose expression is altered by treatment with drug products, chemical entities, or other agents can be identified using a variety of techniques, including gene expression arrays, etc. Reporter cells of the identified genes can be prepared using homologous recombination per above, or the promoters of these genes identified and used in vectors to drive expression of the same marker/reporter. It is understood that the term reporter cells encompasses cells comprising any type of marker gene, including reporter genes, for example. Alternatively, libraries of reporter cells, such as those produced by gene trapping, can be screened with the drug/chemical agent/other agent to identify cells in which marker gene activity is changed. The reporter cells are then used to screen RNAi libraries to identify genes and pathways that regulate expression of the responsive gene. The identified genes in the pathway are then evaluated further using RNAi, gene expression vectors, expression arrays, biochemistry, proteomics, bioinformatics, and other methods to define the direct target of the drug/chemical/agent. Genes in the pathway are both biomarkers and future drug targets.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of reducing nonspecific suppression of gene expression in response to an introduced double-stranded polynucleotide, comprising:
(a) introducing an agent that attenuates a pathway of nonspecific suppression into a cell; and
(b) introducing an RNAi molecule that induces nonspecific suppression of gene expression into the cell,
wherein said agent reduces nonspecific suppression of gene expression induced by said double-stranded polynucleotide.

2. The method of claim 1, wherein the pathway of nonspecific suppression is the PKR pathway.

3. The method of claim 2, wherein the agent alters the activity of a component of the PKR pathway.

4. The method of claim 3, wherein the agent reduces the activity of PKR.

5. The method of claim 3, wherein the agent increases the activity of elongation initiation factor 2a.

6. The method of claim 1, wherein the pathway of nonspecific suppression is the RNase L pathway.

7. The method of claim 1, wherein the agent is a knockout reagent.

8. The method of claim 7, wherein the knockout reagent is selected from the group consisting of: targeting vectors and replacement vectors.

9. The method of claim 1, wherein the agent is a knockdown reagent.

10. The method of claim 9, wherein the knockdown reagent is selected from the group consisting of: antisense RNA; ribozymes; and RNAi molecules.

11. The method of claim 10, wherein the RNAi molecule is selected from the group consisting of: RNA:RNA hybrids, sense DNA:antisense RNA hybrids, sense RNA:antisense DNA hybrids, and DNA:DNA hybrids.

12. The method of claim 1, wherein the agent is a mutant.

13. The method of claim 1, wherein the agent is a dominant negative.

14. The method of claim 1, wherein the RNAi molecule is at least 30 nucleotides in length.

15. The method of claim 1, wherein the RNAi molecule is at least 50 nucleotides in length.

16. The method of claim 1, wherein the RNAi molecule is at least 100 nucleotides in length.

17. The method of claim 1, wherein the RNAi molecule is at least 200 nucleotides in length.

18. The method of claim 1, wherein the RNAi molecule is at least 500 nucleotides in length.

19. The method of claim 1, wherein the RNAI molecule is at least 1000 nucleotides in length.

20. The method of claim 1, wherein the RNAi molecule comprises a full length cDNA sequence.
